SEQUENCE LISTING (126 sequences)

SEQ ID NO: 1 - DNA, 24 bp, Synthetic construct
  source 1..24: mol_type = other DNA
gacgagctctataagtaatctaga

SEQ ID NO: 2 - DNA, 20 bp, Synthetic construct
  source 1..20: mol_type = other DNA
ttttctaggtctctggtcga

SEQ ID NO: 3 - DNA, 53 bp, Synthetic construct
  source 1..53: mol_type = other DNA
ctttccctacacgacgctcttccgatctagacgagctctataagtaatctaga

SEQ ID NO: 4 - DNA, 54 bp, Synthetic construct
  source 1..54: mol_type = other DNA
gtgactggagttcagacgtgtgctcttccgatctttttctaggtctctggtcga

SEQ ID NO: 5 - DNA, 59 bp, Synthetic construct
  source 1..59: mol_type = other DNA
  primer_bind 26..35
aatgatacggcgaccaccgagatctnnnnnnnnnncacactctttccctacacgacgct

SEQ ID NO: 6 - DNA, 54 bp, Synthetic construct
  source 1..54: mol_type = other DNA
  primer_bind 25..34
caagcagaagacggcatacgagatnnnnnnnnnngtgactggagttcagacgtg

SEQ ID NO: 7 - DNA, 56 bp, Synthetic construct
  source 1..56: mol_type = other DNA
gtgactggagttcagacgtgtgctcttccgatctggacgagctctataagtaatct

SEQ ID NO: 8 - DNA, 27 bp, Synthetic construct
  source 1..27: mol_type = other DNA
acactctttccctacacgacgctcttc

SEQ ID NO: 9 - DNA, 51 bp, Synthetic construct
  source 1..51: mol_type = other DNA
  primer_bind 25..34
caagcagaagacggcatacgagatnnnnnnnnnngtgactggagttcagac

SEQ ID NO: 10 - DNA, 52 bp, Synthetic construct
  source 1..52: mol_type = other DNA
  primer_bind 26..35
aatgatacggcgaccaccgagatctnnnnnnnnnnacactctttccctacac

SEQ ID NO: 11 - DNA, 90 bp, Synthetic construct
  source 1..90: mol_type = other DNA
  unsure 31..45
ttcgtataatgtatgctatacgaagttatgnnnnnnnnnnnnnnnnnnnnnnnnngctttaaggccggtcctagcaaaaataaaatacga

SEQ ID NO: 12 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaaggacagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacaacacaaggaacccgcgctataccggtaagctcccgggagcttgtaacaacacgctttaaggccggtcctagcaatttttttaagcctcaataaagcttg

SEQ ID NO: 13 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaaggccagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacaaggtaaggaacccgcgctataccggtaagctcccgggagcttgtaacaaggtgctttaaggccggtcctagcaatttttttaagcctcaataaagcttg

SEQ ID NO: 14 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaaggtcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacactgaaaggaacccgcgctataccggtaagctcccgggagcttgtaacactgagctttaaggccggtcctagcaatttttttaagcctcaataaagcttg

SEQ ID NO: 15 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggaagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacattccaaggaacccgcgctataccggtaagctcccgggagcttgtaacattccgctttaaggccggtcctagcaatttttttaagcctcaataaagcttg

SEQ ID NO: 16 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaaggggagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataaccagccaaggaacccgcgctataccggtaagctcccgggagcttgtaaccagccgctttaaggccggtcctagcaatttttttaagcctcaataaagcttg

SEQ ID NO: 17 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggtagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataaccgtaaaaggaacccgcgctataccggtaagctcccgggagcttgtaaccgtaagctttaaggccggtcctagcaatttttttaagcctcaataaagcttg

SEQ ID NO: 18 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggccgctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacctcttaaggaacccgcgctataccggtaagctcccgggagcttgtaacctcttgctttaaggccggtcctagcaatttttttaagcctcaataaagcttg

SEQ ID NO: 19 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcggctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacgagaaaaggaacccgcgctataccggtaagctcccgggagcttgtaacgagaagctttaaggccggtcctagcaatttttttaagcctcaataaagcttg

SEQ ID NO: 20 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggctgctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacgtcgcaaggaacccgcgctataccggtaagctcccgggagcttgtaacgtcgcgctttaaggccggtcctagcaatttttttaagcctcaataaagcttg

SEQ ID NO: 21 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcaactggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataacttggaaaggaacccgcgctataccggtaagctcccgggagcttgtaacttggagctttaaggccggtcctagcaatttttttaagcctcaataaagcttg

SEQ ID NO: 22 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcacctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataagaatcgaaggaacccgcgctataccggtaagctcccgggagcttgtaagaatcggctttaaggccggtcctagcaatttttttaagcctcaataaagcttg

SEQ ID NO: 23 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcatctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataagaccaaaaggaacccgcgctataccggtaagctcccgggagcttgtaagaccaagctttaaggccggtcctagcaatttttttaagcctcaataaagcttg

SEQ ID NO: 24 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagatggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataagcggtgaaggaacccgcgctataccggtaagctcccgggagcttgtaagcggtggctttaaggccggtcctagcaatttttttaagcctcaataaagcttg

SEQ ID NO: 25 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcaggtggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataagctacgaaggaacccgcgctataccggtaagctcccgggagcttgtaagctacggctttaaggccggtcctagcaatttttttaagcctcaataaagcttg

SEQ ID NO: 26 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagttggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataaggagctaaggaacccgcgctataccggtaagctcccgggagcttgtaaggagctgctttaaggccggtcctagcaatttttttaagcctcaataaagcttg

SEQ ID NO: 27 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagcaggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataaggtcataaggaacccgcgctataccggtaagctcccgggagcttgtaaggtcatgctttaaggccggtcctagcaatttttttaagcctcaataaagcttg

SEQ ID NO: 28 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagccggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataataccgcaaggaacccgcgctataccggtaagctcccgggagcttgtaataccgcgctttaaggccggtcctagcaatttttttaagcctcaataaagcttg

SEQ ID NO: 29 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagcgggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataatagtggaaggaacccgcgctataccggtaagctcccgggagcttgtaatagtgggctttaaggccggtcctagcaatttttttaagcctcaataaagcttg

SEQ ID NO: 30 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctagtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataatctccaaaggaacccgcgctataccggtaagctcccgggagcttgtaatctccagctttaaggccggtcctagcaatttttttaagcctcaataaagcttg

SEQ ID NO: 31 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctcgtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataatgccttaaggaacccgcgctataccggtaagctcccgggagcttgtaatgccttgctttaaggccggtcctagcaatttttttaagcctcaataaagcttg

SEQ ID NO: 32 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagcttgtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacaacttcaaggaacccgcgctataccggtaagctcccgggagcttgtacaacttcgctttaaggccggtcctagcaatttttttaagcctcaataaagcttg

SEQ ID NO: 33 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctgatcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacaatagcaaggaacccgcgctataccggtaagctcccgggagcttgtacaatagcgctttaaggccggtcctagcaatttttttaagcctcaataaagcttg

SEQ ID NO: 34 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctgctcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacacgcaaaaggaacccgcgctataccggtaagctcccgggagcttgtacacgcaagctttaaggccggtcctagcaatttttttaagcctcaataaagcttg

SEQ ID NO: 35 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctgttcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacaggattaaggaacccgcgctataccggtaagctcccgggagcttgtacaggattgctttaaggccggtcctagcaatttttttaagcctcaataaagcttg

SEQ ID NO: 36 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcaaatggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataccacagtaaggaacccgcgctataccggtaagctcccgggagcttgtaccacagtgctttaaggccggtcctagcaatttttttaagcctcaataaagcttg

SEQ ID NO: 37 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcaattggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataccgacctaaggaacccgcgctataccggtaagctcccgggagcttgtaccgacctgctttaaggccggtcctagcaatttttttaagcctcaataaagcttg

SEQ ID NO: 38 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcaagtggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataccgtgtaaaggaacccgcgctataccggtaagctcccgggagcttgtaccgtgtagctttaaggccggtcctagcaatttttttaagcctcaataaagcttg

SEQ ID NO: 39 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcatatggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacctagataaggaacccgcgctataccggtaagctcccgggagcttgtacctagatgctttaaggccggtcctagcaatttttttaagcctcaataaagcttg

SEQ ID NO: 40 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcatttggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataccttgccaaggaacccgcgctataccggtaagctcccgggagcttgtaccttgccgctttaaggccggtcctagcaatttttttaagcctcaataaagcttg

SEQ ID NO: 41 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcatgtggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacgagtccaaggaacccgcgctataccggtaagctcccgggagcttgtacgagtccgctttaaggccggtcctagcaatttttttaagcctcaataaagcttg

SEQ ID NO: 42 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcacatggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacgcataaaaggaacccgcgctataccggtaagctcccgggagcttgtacgcataagctttaaggccggtcctagcaatttttttaagcctcaataaagcttg

SEQ ID NO: 43 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcacttggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacggacgaaaggaacccgcgctataccggtaagctcccgggagcttgtacggacgagctttaaggccggtcctagcaatttttttaagcctcaataaagcttg

SEQ ID NO: 44 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcacgtggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacgtatggaaggaacccgcgctataccggtaagctcccgggagcttgtacgtatgggctttaaggccggtcctagcaatttttttaagcctcaataaagcttg

SEQ ID NO: 45 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaacttggaacaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacgttcaaaaggaacccgcgctataccggtaagctcccgggagcttgtacgttcaagctttaaggccggtcctagcaatttttttaagcctcaataaagcttg

SEQ ID NO: 46 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcggcaccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatactaaccaaaggaacccgcgctataccggtaagctcccgggagcttgtactaaccagctttaaggccggtcctagcaatttttttaagcctcaataaagcttg

SEQ ID NO: 47 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggccacagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatactatctgaaggaacccgcgctataccggtaagctcccgggagcttgtactatctggctttaaggccggtcctagcaatttttttaagcctcaataaagcttg

SEQ ID NO: 48 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaaagcgggctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatactcaggtaaggaacccgcgctataccggtaagctcccgggagcttgtactcaggtgctttaaggccggtcctagcaatttttttaagcctcaataaagcttg

SEQ ID NO: 49 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcacggtgtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatactccgaaaaggaacccgcgctataccggtaagctcccgggagcttgtactccgaagctttaaggccggtcctagcaatttttttaagcctcaataaagcttg

SEQ ID NO: 50 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctgggggtcgcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatacttgttgaaggaacccgcgctataccggtaagctcccgggagcttgtacttgttggctttaaggccggtcctagcaatttttttaagcctcaataaagcttg

SEQ ID NO: 51 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcgggggttctcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagaacagaaaggaacccgcgctataccggtaagctcccgggagcttgtagaacagagctttaaggccggtcctagcaatttttttaagcctcaataaagcttg

SEQ ID NO: 52 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttggcagtaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagaagtacaaggaacccgcgctataccggtaagctcccgggagcttgtagaagtacgctttaaggccggtcctagcaatttttttaagcctcaataaagcttg

SEQ ID NO: 53 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaagaaggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagacgcttaaggaacccgcgctataccggtaagctcccgggagcttgtagacgcttgctttaaggccggtcctagcaatttttttaagcctcaataaagcttg

SEQ ID NO: 54 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggatattcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagagatgaaaggaacccgcgctataccggtaagctcccgggagcttgtagagatgagctttaaggccggtcctagcaatttttttaagcctcaataaagcttg

SEQ ID NO: 55 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatccgtctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagatgcgaaaggaacccgcgctataccggtaagctcccgggagcttgtagatgcgagctttaaggccggtcctagcaatttttttaagcctcaataaagcttg

SEQ ID NO: 56 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgcactgcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagattaggaaggaacccgcgctataccggtaagctcccgggagcttgtagattagggctttaaggccggtcctagcaatttttttaagcctcaataaagcttg

SEQ ID NO: 57 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagccctcataatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagccactcaaggaacccgcgctataccggtaagctcccgggagcttgtagccactcgctttaaggccggtcctagcaatttttttaagcctcaataaagcttg

SEQ ID NO: 58 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatcacttatcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagcctggtaaggaacccgcgctataccggtaagctcccgggagcttgtagcctggtgctttaaggccggtcctagcaatttttttaagcctcaataaagcttg

SEQ ID NO: 59 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctgccattctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagcgaagcaaggaacccgcgctataccggtaagctcccgggagcttgtagcgaagcgctttaaggccggtcctagcaatttttttaagcctcaataaagcttg

SEQ ID NO: 60 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagctttgccttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagctctaaaaggaacccgcgctataccggtaagctcccgggagcttgtagctctaagctttaaggccggtcctagcaatttttttaagcctcaataaagcttg

SEQ ID NO: 61 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctggctgtaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataggtaacgaaggaacccgcgctataccggtaagctcccgggagcttgtaggtaacggctttaaggccggtcctagcaatttttttaagcctcaataaagcttg

SEQ ID NO: 62 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggtccacccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgataggtgtctaaggaacccgcgctataccggtaagctcccgggagcttgtaggtgtctgctttaaggccggtcctagcaatttttttaagcctcaataaagcttg

SEQ ID NO: 63 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctacccccccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatagtacatcaaggaacccgcgctataccggtaagctcccgggagcttgtagtacatcgctttaaggccggtcctagcaatttttttaagcctcaataaagcttg

SEQ ID NO: 64 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgactttcttccccaaacgcaaccagtaatcctcacggcttgtctctgatccgtgggctgatgctcttatctctgctggcggactgaccgtggaagcactgtacaagtaactgcgatagtccgttaaggaacccgcgctataccggtaagctcccgggagcttgtagtccgttgctttaaggccggtcctagcaatttttttaagcctcaataaagcttg

SEQ ID NO: 65 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctatctccccaaacgcaaccagtaatcctcacggcttgtctctgatccgtgggctgatgctcttatctctgctggcggactgaccgtggaagcactgtacaagtaactgcgatagtgattcaaggaacccgcgctataccggtaagctcccgggagcttgtagtgattcgctttaaggccggtcctagcaatttttttaagcctcaataaagcttg

SEQ ID NO: 66 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccaaacccgcaaccagtaatcctcacggcttgtctctgatccgtgggctgatgctcttatctctgctggcggactgaccgtggaagcactgtacaagtaactgcgatagttcgcaaaggaacccgcgctataccggtaagctcccgggagcttgtagttcgcagctttaaggccggtcctagcaatttttttaagcctcaataaagcttg

SEQ ID NO: 67 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaacgctaatcctcacggcttgtctctgatccgtgggctgatgctcttatctctgctggcggactgaccgtggaagcactgtacaagtaactgcgatataagaggaaggaacccgcgctataccggtaagctcccgggagcttgtataagagggctttaaggccggtcctagcaatttttttaagcctcaataaagcttg

SEQ ID NO: 68 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtcaatctcacggcttgtctctgatccgtgggctgatgctcttatctctgctggcggactgaccgtggaagcactgtacaagtaactgcgatataagctcaaggaacccgcgctataccggtaagctcccgggagcttgtataagctcgctttaaggccggtcctagcaatttttttaagcctcaataaagcttg

SEQ ID NO: 69 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatccgctcagcttgtctctgatccgtgggctgatgctcttatctctgctggcggactgaccgtggaagcactgtacaagtaactgcgatatatcacgaaggaacccgcgctataccggtaagctcccgggagcttgtatatcacggctttaaggccggtcctagcaatttttttaagcctcaataaagcttg

SEQ ID NO: 70 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacgttgcgtctctgatccgtgggctgatgctcttatctctgctggcggactgaccgtggaagcactgtacaagtaactgcgatatccatgaaaggaacccgcgctataccggtaagctcccgggagcttgtatccatgagctttaaggccggtcctagcaatttttttaagcctcaataaagcttg

SEQ ID NO: 71 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgttctcgatccgtgggctgatgctcttatctctgctggcggactgaccgtggaagcactgtacaagtaactgcgatatcgccgtaaggaacccgcgctataccggtaagctcccgggagcttgtatcgccgtgctttaaggccggtcctagcaatttttttaagcctcaataaagcttg

SEQ ID NO: 72 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctctcgagtgggctgatgctcttatctctgctggcggactgaccgtggaagcactgtacaagtaactgcgatatctagcgaaggaacccgcgctataccggtaagctcccgggagcttgtatctagcggctttaaggccggtcctagcaatttttttaagcctcaataaagcttg

SEQ ID NO: 73 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccggggtctgatgctcttatctctgctggcggactgaccgtggaagcactgtacaagtaactgcgatatgacggaaaggaacccgcgctataccggtaagctcccgggagcttgtatgacggagctttaaggccggtcctagcaatttttttaagcctcaataaagcttg

SEQ ID NO: 74 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggcttgagctcttatctctgctggcggactgaccgtggaagcactgtacaagtaactgcgatatgcaaccaaggaacccgcgctataccggtaagctcccgggagcttgtatgcaaccgctttaaggccggtcctagcaatttttttaagcctcaataaagcttg

SEQ ID NO: 75 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatcgtcttatctctgctggcggactgaccgtggaagcactgtacaagtaactgcgatatggaatgaaggaacccgcgctataccggtaagctcccgggagcttgtatggaatggctttaaggccggtcctagcaatttttttaagcctcaataaagcttg

SEQ ID NO: 76 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctctatttcctgctggcggactgaccgtggaagcactgtacaagtaactgcgatatgtgcagaaggaacccgcgctataccggtaagctcccgggagcttgtatgtgcaggctttaaggccggtcctagcaatttttttaagcctcaataaagcttg

SEQ ID NO: 77 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctgtctcggcggactgaccgtggaagcactgtacaagtaactgcgatattcctacaaggaacccgcgctataccggtaagctcccgggagcttgtattcctacgctttaaggccggtcctagcaatttttttaagcctcaataaagcttg

SEQ ID NO: 78 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctcggggactgaccgtggaagcactgtacaagtaactgcgatattggtagaaggaacccgcgctataccggtaagctcccgggagcttgtattggtaggctttaaggccggtcctagcaatttttttaagcctcaataaagcttg

SEQ ID NO: 79 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggatcgaccgtggaagcactgtacaagtaactgcgatcaacgccaaaggaacccgcgctataccggtaagctcccgggagcttgtcaacgccagctttaaggccggtcctagcaatttttttaagcctcaataaagcttg

SEQ ID NO: 80 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaatccctgggctgatgctcttatctctgctggcggactgaccgtggaagcactgtacaagtaactgcgatcaagaagaaaggaacccgcgctataccggtaagctcccgggagcttgtcaagaagagctttaaggccggtcctagcaatttttttaagcctcaataaagcttg

SEQ ID NO: 81 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaatccccacggcttgtctctgatccgtgggctgatgctcttatctctgctggcggactgaccgtggaagcactgtacaagtaactgcgatcaagtctgaaggaacccgcgctataccggtaagctcccgggagcttgtcaagtctggctttaaggccggtcctagcaatttttttaagcctcaataaagcttg

SEQ ID NO: 82 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcaatggacaaggaacccgcgctataccggtaagctcccgggagcttgtcaatggacgctttaaggccggtcctagcaatttttttaagcctcaataaagcttg

SEQ ID NO: 83 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcacacatcaaggaacccgcgctataccggtaagctcccgggagcttgtcacacatcgctttaaggccggtcctagcaatttttttaagcctcaataaagcttg

SEQ ID NO: 84 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggattaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcacatgctaaggaacccgcgctataccggtaagctcccgggagcttgtcacatgctgctttaaggccggtcctagcaatttttttaagcctcaataaagcttg

SEQ ID NO: 85 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaagccctgggctgatgctcttatctctgctggcggactgaccgtggaagcactgtacaagtaactgcgatcaccttataaggaacccgcgctataccggtaagctcccgggagcttgtcaccttatgctttaaggccggtcctagcaatttttttaagcctcaataaagcttg

SEQ ID NO: 86 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaagccccacggcttgtctctgatccgtgggctgatgctcttatctctgctggcggactgaccgtggaagcactgtacaagtaactgcgatcacggtagaaggaacccgcgctataccggtaagctcccgggagcttgtcacggtaggctttaaggccggtcctagcaatttttttaagcctcaataaagcttg

SEQ ID NO: 87 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaagcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcagaacctaaggaacccgcgctataccggtaagctcccgggagcttgtcagaacctgctttaaggccggtcctagcaatttttttaagcctcaataaagcttg

SEQ ID NO: 88 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggcttagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcagaggttaaggaacccgcgctataccggtaagctcccgggagcttgtcagaggttgctttaaggccggtcctagcaatttttttaagcctcaataaagcttg

SEQ ID NO: 89 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggcttaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcagccgataaggaacccgcgctataccggtaagctcccgggagcttgtcagccgatgctttaaggccggtcctagcaatttttttaagcctcaataaagcttg

SEQ ID NO: 90 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctcttatccctgggctgatgctcttatctctgctggcggactgaccgtggaagcactgtacaagtaactgcgatcagtatagaaggaacccgcgctataccggtaagctcccgggagcttgtcagtataggctttaaggccggtcctagcaatttttttaagcctcaataaagcttg

SEQ ID NO: 91 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccacttatccccacggcttgtctctgatccgtgggctgatgctcttatctctgctggcggactgaccgtggaagcactgtacaagtaactgcgatcatactgtaaggaacccgcgctataccggtaagctcccgggagcttgtcatactgtgctttaaggccggtcctagcaatttttttaagcctcaataaagcttg

SEQ ID NO: 92 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatcttatcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcatcaagtaaggaacccgcgctataccggtaagctcccgggagcttgtcatcaagtgctttaaggccggtcctagcaatttttttaagcctcaataaagcttg

SEQ ID NO: 93 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggataagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcatccaccaaggaacccgcgctataccggtaagctcccgggagcttgtcatccaccgctttaaggccggtcctagcaatttttttaagcctcaataaagcttg

SEQ ID NO: 94 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggataaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcatgttccaaggaacccgcgctataccggtaagctcccgggagcttgtcatgttccgctttaaggccggtcctagcaatttttttaagcctcaataaagcttg

SEQ ID NO: 95 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctactccctgggctgatgctcttatctctgctggcggactgaccgtggaagcactgtacaagtaactgcgatcattgagcaaggaacccgcgctataccggtaagctcccgggagcttgtcattgagcgctttaaggccggtcctagcaatttttttaagcctcaataaagcttg

SEQ ID NO: 96 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactactccccacggcttgtctctgatccgtgggctgatgctcttatctctgctggcggactgaccgtggaagcactgtacaagtaactgcgatccaacaataaggaacccgcgctataccggtaagctcccgggagcttgtccaacaatgctttaaggccggtcctagcaatttttttaagcctcaataaagcttg

SEQ ID NO: 97 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctactcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatccaagcgtaaggaacccgcgctataccggtaagctcccgggagcttgtccaagcgtgctttaaggccggtcctagcaatttttttaagcctcaataaagcttg

SEQ ID NO: 98 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggagtagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatccaattacaaggaacccgcgctataccggtaagctcccgggagcttgtccaattacgctttaaggccggtcctagcaatttttttaagcctcaataaagcttg

SEQ ID NO: 99 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggagtaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatccacgactaaggaacccgcgctataccggtaagctcccgggagcttgtccacgactgctttaaggccggtcctagcaatttttttaagcctcaataaagcttg

SEQ ID NO: 100 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactactccccacggcttgtctctactccctgggctgatgctcttatctctgctggcggactgaccgtggaagcactgtacaagtaactgcgatccagtgaaaaggaacccgcgctataccggtaagctcccgggagcttgtccagtgaagctttaaggccggtcctagcaatttttttaagcctcaataaagcttg

SEQ ID NO: 101 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctactcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctactccctgggctgatgctcttatctctgctggcggactgaccgtggaagcactgtacaagtaactgcgatccattgtcaaggaacccgcgctataccggtaagctcccgggagcttgtccattgtcgctttaaggccggtcctagcaatttttttaagcctcaataaagcttg

SEQ ID NO: 102 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggagtagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctactccctgggctgatgctcttatctctgctggcggactgaccgtggaagcactgtacaagtaactgcgatccgatcagaaggaacccgcgctataccggtaagctcccgggagcttgtccgatcaggctttaaggccggtcctagcaatttttttaagcctcaataaagcttg

SEQ ID NO: 103 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggagtaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctactccctgggctgatgctcttatctctgctggcggactgaccgtggaagcactgtacaagtaactgcgatccgcatgtaaggaacccgcgctataccggtaagctcccgggagcttgtccgcatgtgctttaaggccggtcctagcaatttttttaagcctcaataaagcttg

SEQ ID NO: 104 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctactcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactactccccacggcttgtctctgatccgtgggctgatgctcttatctctgctggcggactgaccgtggaagcactgtacaagtaactgcgatccggtcttaaggaacccgcgctataccggtaagctcccgggagcttgtccggtcttgctttaaggccggtcctagcaatttttttaagcctcaataaagcttg

SEQ ID NO: 105 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggagtagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactactccccacggcttgtctctgatccgtgggctgatgctcttatctctgctggcggactgaccgtggaagcactgtacaagtaactgcgatcctactccaaggaacccgcgctataccggtaagctcccgggagcttgtcctactccgctttaaggccggtcctagcaatttttttaagcctcaataaagcttg

SEQ ID NO: 106 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggagtaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactactccccacggcttgtctctgatccgtgggctgatgctcttatctctgctggcggactgaccgtggaagcactgtacaagtaactgcgatcctcatacaaggaacccgcgctataccggtaagctcccgggagcttgtcctcatacgctttaaggccggtcctagcaatttttttaagcctcaataaagcttg

SEQ ID NO: 107 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggagtagcctgctcagcccatctactcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcctccttgaaggaacccgcgctataccggtaagctcccgggagcttgtcctccttggctttaaggccggtcctagcaatttttttaagcctcaataaagcttg

SEQ ID NO: 108 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggagtaggcccaagggcagctggtcggggcttgtcgagaaaagggattatcctgctcagcccatctactcccagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcctgagttaaggaacccgcgctataccggtaagctcccgggagcttgtcctgagttgctttaaggccggtcctagcaatttttttaagcctcaataaagcttg

SEQ ID NO: 109 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgggagtaggcccaagggcagctggtcggggcttgtcgagaaaagggagtagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatccttaatgaaggaacccgcgctataccggtaagctcccgggagcttgtccttaatggctttaaggccggtcctagcaatttttttaagcctcaataaagcttg

SEQ ID NO: 110 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaattactgggctgatgctcttatctctgctggcggactgaccgtggaagcactgtacaagtaactgcgatccttcggaaaggaacccgcgctataccggtaagctcccgggagcttgtccttcggagctttaaggccggtcctagcaatttttttaagcctcaataaagcttg

SEQ ID NO: 111 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaattaccacggcttgtctctgatccgtgggctgatgctcttatctctgctggcggactgaccgtggaagcactgtacaagtaactgcgatcgaatatcaaggaacccgcgctataccggtaagctcccgggagcttgtcgaatatcgctttaaggccggtcctagcaatttttttaagcctcaataaagcttg

SEQ ID NO: 112 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaattacagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgacaacgaaggaacccgcgctataccggtaagctcccgggagcttgtcgacaacggctttaaggccggtcctagcaatttttttaagcctcaataaagcttg

SEQ ID NO: 113 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagtaattagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgagagcaaaggaacccgcgctataccggtaagctcccgggagcttgtcgagagcagctttaaggccggtcctagcaatttttttaagcctcaataaagcttg

SEQ ID NO: 114 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgtaattaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgccagtaaaggaacccgcgctataccggtaagctcccgggagcttgtcgccagtagctttaaggccggtcctagcaatttttttaagcctcaataaagcttg

SEQ ID NO: 115 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaattaccacggcttgtctctaattactgggctgatgctcttatctctgctggcggactgaccgtggaagcactgtacaagtaactgcgatcgcctcaaaaggaacccgcgctataccggtaagctcccgggagcttgtcgcctcaagctttaaggccggtcctagcaatttttttaagcctcaataaagcttg

SEQ ID NO: 116 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaattacagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaattactgggctgatgctcttatctctgctggcggactgaccgtggaagcactgtacaagtaactgcgatcgcggaataaggaacccgcgctataccggtaagctcccgggagcttgtcgcggaatgctttaaggccggtcctagcaatttttttaagcctcaataaagcttg

SEQ ID NO: 117 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagtaattagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaattactgggctgatgctcttatctctgctggcggactgaccgtggaagcactgtacaagtaactgcgatcgcgttacaaggaacccgcgctataccggtaagctcccgggagcttgtcgcgttacgctttaaggccggtcctagcaatttttttaagcctcaataaagcttg

SEQ ID NO: 118 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgtaattaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctaattactgggctgatgctcttatctctgctggcggactgaccgtggaagcactgtacaagtaactgcgatcggaaggaaaggaacccgcgctataccggtaagctcccgggagcttgtcggaaggagctttaaggccggtcctagcaatttttttaagcctcaataaagcttg

SEQ ID NO: 119 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaattacagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaattaccacggcttgtctctgatccgtgggctgatgctcttatctctgctggcggactgaccgtggaagcactgtacaagtaactgcgatcggactctaaggaacccgcgctataccggtaagctcccgggagcttgtcggactctgctttaaggccggtcctagcaatttttttaagcctcaataaagcttg

SEQ ID NO: 120 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagtaattagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaattaccacggcttgtctctgatccgtgggctgatgctcttatctctgctggcggactgaccgtggaagcactgtacaagtaactgcgatcggtgagaaaggaacccgcgctataccggtaagctcccgggagcttgtcggtgagagctttaaggccggtcctagcaatttttttaagcctcaataaagcttg

SEQ ID NO: 121 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgtaattaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccactaattaccacggcttgtctctgatccgtgggctgatgctcttatctctgctggcggactgaccgtggaagcactgtacaagtaactgcgatcggttgttaaggaacccgcgctataccggtaagctcccgggagcttgtcggttgttgctttaaggccggtcctagcaatttttttaagcctcaataaagcttg

SEQ ID NO: 122 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagtaattagcctgctcagcccatctaattacagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgtaacacaaggaacccgcgctataccggtaagctcccgggagcttgtcgtaacacgctttaaggccggtcctagcaatttttttaagcctcaataaagcttg

SEQ ID NO: 123 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgtaattaggcccaagggcagctggtcggggcttgtcgagaaaagggattatcctgctcagcccatctaattacagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgtagcttaaggaacccgcgctataccggtaagctcccgggagcttgtcgtagcttgctttaaggccggtcctagcaatttttttaagcctcaataaagcttg

SEQ ID NO: 124 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactgtaattaggcccaagggcagctggtcggggcttgtcgagaaaagtaattagcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgtctatgaaggaacccgcgctataccggtaagctcccgggagcttgtcgtctatggctttaaggccggtcctagcaatttttttaagcctcaataaagcttg

SEQ ID NO: 125 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatcgttctcgaaggaacccgcgctataccggtaagctcccgggagcttgtcgttctcggctttaaggccggtcctagcaatttttttaagcctcaataaagcttg

SEQ ID NO: 126 - DNA, 661 bp, Synthetic construct
  source 1..661: mol_type = other DNA
gtgcaccatagccagatataaagaccctgggagtcatgtgtggatgcatgaggacagaccccagccgagaagcaggacacagagctctgttctcttttgtagtcatttctttgcccacaacacaggggcacatgggcccagctccccccacccccatctccctgtcaactcccccaactaggatcaggcccaagggcagctggtcggggcttgtcgagaaaagggattatcctgctcagcccatctaatctcagcttctgcttggaccctccccacccctccccacagccctcaacgaggaagcatggtttgtagtgttgacaacagggtcatctgtcaccctcctgttctggctcctggcccgcttgctgaacaaggccaagatcatgaccacaggttggggctagggcctggggaggtcactggagctatgactgctcctgttctacttccccaaacgcaaccagtaatcctcacggcttgtctctgatccgtgggctgatgctcttatctctgctggcggactgaccgtggaagcactgtacaagtaactgcgatctaccggaaaggaacccgcgctataccggtaagctcccgggagcttgtctaccggagctttaaggccggtcctagcaatttttttaagcctcaataaagcttg

(19) United States
(12) Patent Application Publication
(10) Pub. No.: US 2024/0026345 A1
(43) Pub. Date: Jan. 25, 2024

(54) PARALLEL SINGLE-CELL REPORTER ASSAYS AND COMPOSITIONS

(71) Applicant: Washington University, St. Louis, MO (US)

(72) Inventors: Barak Cohen, St. Louis, MO (US); Siqi Zhao, St. Louis, MO (US)

(73) Assignee: Washington University, St. Louis, MO (US)

(21) Appl. No.: 18/054,503

(22) Filed: Nov. 10, 2022

Related U.S. Application Data
(60) Provisional application No. 63/391,404, filed on Jul. 22, 2022.

(51) Int. Cl. C12N 15/10; C12N 15/86

(52) U.S. Cl. CPC C12N 15/1037; C12N 15/86; C12N 2750/14143

(57) ABSTRACT

Among the various aspects of the present disclosure is the provision of compositions for single-cell reporter assays and methods of use thereof. Also provided are methods of determining individual activities of a plurality of nucleic acid regulatory elements, identifying a regulatory element having cell type-specific activity, or determining variance in activity of a plurality of nucleic acid regulatory elements.

### CROSS-REFERENCE TO RELATED APPLICATIONS

This application claims priority from U.S. Provisional Application Ser. No. 63/391,404 filed on Jul. 22, 2022, which is incorporated herein by reference in its entirety. This application also claims priority from U.S. Provisional Application Ser. No. 63/277,950 filed on Nov. 10, 2021, which is incorporated herein by reference in its entirety.

### STATEMENT REGARDING FEDERALLY SPONSORED RESEARCH OR DEVELOPMENT

This invention was made with government support under GM092910 and GM140711 awarded by the National Institutes of Health. The government has certain rights in the invention.

### MATERIAL INCORPORATED-BY-REFERENCE

The Sequence Listing (019931-US-NP_SEQ_LIST_SUBSTITUTE.XML, 10,240 bytes, generated 6/20/2023), which is a part of the present disclosure, includes a computer-readable form comprising nucleotide and/or amino acid sequences of the present invention. The subject matter of the Sequence Listing is incorporated herein by reference in its entirety.

### FIELD

The present disclosure generally relates to methods of using Massively Parallel Reporter Assays (MPRAs) in single cells.

### BACKGROUND

The majority of heritable variation for human diseases maps to the non-coding portions of the genome. This observation has led to the hypothesis that genetic variation in the cis-regulatory sequences (CRSs) that control gene expression underlies a large fraction of disease burden. Because many CRSs function only in specific cell types, there is intense interest in high-throughput assays that can measure the effects of cell-type-specific CRSs and their genetic variants.

Massively Parallel Reporter Assays (MPRAs) are one family of techniques that allow investigators to assay libraries of CRSs and their non-coding variants en masse. In an MPRA experiment, every CRS drives a reporter gene carrying a unique DNA barcode in its 3′ UTR, which allows investigators to quantify the activity of each CRS by the ratio of its barcode abundances in the output RNA and input DNA. This approach allows investigators to identify new CRSs, assay the effects of non-coding variants, and discover general rules governing the functions of CRSs. One limitation of MPRAs is that they are generally performed in monocultures, or as bulk assays across the cell types of a tissue. Performing cell-type specific MPRAs in tissues will require methods to simultaneously readout reporter gene activities and cell type information in heterogeneous pools of cells.

### SUMMARY

Among the various aspects of the present disclosure are the provision of a composition for single-cell Massively Parallel Reporter Assays and methods of use thereof.

In one aspect, the present disclosure provides for a plurality of expression vectors, wherein an individual expression vector comprises a first identifying nucleic acid barcode (rBC) uniquely associated with the individual expression vector. In some aspects, the first identifying nucleic acid barcode (rBC) is a randomized sequence. In some aspects, each expression vector further comprises a nucleic acid regulatory element; an open reading frame optionally encoding a reporter gene; and a second identifying nucleic acid barcode (cBC) uniquely associated with the nucleic acid regulatory element; wherein the nucleic acid regulatory element of each expression vector is selected from a plurality of different nucleic acid regulatory elements. In some aspects, each nucleic acid regulatory element is a genetic variant of a single nucleic acid regulatory element. In some aspects, each nucleic acid regulatory element differs from the remaining nucleic acid regulatory elements by at least one nucleotide substitution, deletion, or insertion. In some aspects, the regulatory element is a cis-regulatory element. In some aspects, the cis-regulatory element is an enhancer, promoter, insulator, or silencer. In some aspects, the cis-regulatory element is a core promoter. In some aspects, each expression vector further comprises a cell barcode or a UMI sequence. In some aspects, the cell barcode comprises a 10× cell barcode and the UMI sequence comprises a 10× UMI sequence. In some aspects, each expression vector further comprises a capture sequence or a polyadenylation signal. In some aspects, the nucleic acid regulatory element and the cBC are linked. In some aspects, the nucleic acid regulatory element and cBC are linked through a process selected from synthesis, ligation, PCR, and any combination thereof.

In another aspect, the present disclosure provides for a method for determining individual activities of a plurality of nucleic acid regulatory elements, the method comprising introducing the plurality of expression vectors into a population of cells; performing single-cell RNA sequencing (scRNAseq) on the population of cells; and quantifying expression of cBC and/or rBC in an individual cell; wherein the amount of each cBC detected indicates the activity of the associated regulatory element in the cell; and the amount of each rBC detected indicates the number of expression vectors comprising the associated regulatory element in the cell. In some aspects, the method further comprises generating a scRNAseq profile for the individual cell, wherein the scRNAseq profile identifies the cell type of the individual cell. In some aspects, the population of cells comprises cells in different biological states, the different biological states comprise different stages of cell cycle, different subpopulations of same cell type, or a combination thereof. In some aspects, the population of cells comprises multiple cell types. In some aspects, the method further comprises normalizing the activity of the regulatory element to the number of expression vectors comprising the regulatory element in the cell.

In yet another aspect, the present disclosure provides for a method for identifying a regulatory element having cell type-specific activity, the method comprising introducing the plurality of expression vectors into a population of cells; performing single-cell RNA sequencing (scRNAseq) on the population of cells; quantifying expression of cBC and/or rBC in an individual cell; wherein the amount of each cBC detected indicates the activity of the associated regulatory element in the cell; and the amount of each rBC detected indicates the number of expression vectors comprising the associated regulatory element in the cell; generating a scRNAseq profile for the individual cell, wherein the scRNAseq profile identifies the cell type of the individual cell; determining the regulatory element to have cell type-specific activity if the activity of the regulatory element differs substantially between at least two cell types.

In yet another aspect, the present disclosure provides for a method for determining variance in activity of a plurality of nucleic acid regulatory elements, the method comprising introducing the plurality of expression vectors into a population of cells; performing single-cell RNA sequencing (scRNAseq) on the population of cells; quantifying expression of cBC and/or rBC in an individual cell; wherein the amount of each cBC detected indicates the activity of the associated regulatory element in the cell; and the amount of each rBC detected indicates the number of expression vectors comprising the associated regulatory element in the cell; and calculating the variance in activity of the regulatory element across the population of cells.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration showing an overview of the single cell Massively Parallel Reporter Assays (scMPRA) described in the present disclosure.

FIG. 2 is a schematic illustration showing how the complex barcoding strategy described in the present disclosure enables input plasm id normalization.

FIG. 3 is a schematic illustration showing double-capturing sequences used to facilitate efficient capturing.

FIG. 4 is a set of UMAPs and graphs showing the method of the current disclosure can detect cell-type specific activity in mixed cells.

FIG. 5 contains a pair of plots showing the identification of cell-cycle specific CRS expression using scMPRA.

FIG. 6 is a set of UMAPs and a genetic heat map showing the identification of CRS-specific expression in rare populations in cancer cell lines using scMPRA.

FIG. 7A is a schematic illustration of a CRS reporter construct. Each CRS reporter construct is barcoded with a cBC that specifies the identity of the CRS, and a highly complex rBC. The complexity of the cBC-rBC pair ensures that the probability of identical plasm ids being introduced into the same cell is extremely low.

FIG. 7B is a schematic illustration of the experimental overview for scMPRA using the mixed-cell experiment as an example. K562 cells and HEK293 cells are transfected with the double-barcoded core promoter library. After 24 hours, cells were harvested and mixed for 10× scRNA-seq. Cell identities were obtained by sequencing the transcriptome, and single-cell expression of CRSs was obtained by quantifying the barcodes. The cell identity and CRSs expression (as measured by the cBC-rBC abundances) were linked by the shared 10× cell barcodes.

FIG. 8A is a UMAP of the transcriptome from the mixed-cell scMPRA experiment. 3312 out of 3417 cells are assigned to either K562 or HEK293 cells and visualised here.

FIG. 8B is a graph of reproducibility of replicate measurements of the mean expression from each core promoter in K562 cells.

FIG. 8C is a graph of reproducibility of replicate measurements of the mean expression from each core promoter in HEK293 cells.

FIG. 8D is a histogram of the number of cells in which each core promoter was measured for K562 cells.

FIG. 8E is a histogram of the number of cells in which each core promoter was measured for HEK293 cells.

FIG. 8F is a graph showing correlations between scMPRA and bulk MPRA in K562 cells using mRNA abundances (cBC counts per cell) to make the two methods comparable.

FIG. 8G is a graph showing correlations between scMPRA and bulk MPRA in HEK293 cells using mRNA abundances (cBC counts per cell) to make the two methods comparable.

FIG. 8H is a boxplot of the activities of core promoters from different categories in K562 (orange) and HEK293 (blue) cells. Because the average expressions of all promoters were different between K562 and HEK293, each category was plotted according to its deviation from the average expression (z-score) of all promoters in each cell type.

FIG. 8I is a volcano plot for differential expression (DE) of core promoters in K562 and HEK293 cells. Red dots represent significant DE reporters (two-sided Wald test adjusted p-value <0.01 and log 2-fold change greater than 0.3).

FIG. 8J is a Venn diagram of the functional characterization (housekeeping vs developmental) of down-regulated core promoters in K562 cells. Housekeeping promoters are enriched (p-value=1.08×10⁻¹¹ from two-sided hypergeometric test).

FIG. 8K is a pie chart of the sequence features (CpG, DPE, TATA) of down-regulated core promoters. CpG promoters are enriched (p=2.18×10⁻⁶, two-sided hypergeometric test).

FIG. 9A is a PCA plot of K562 cells classified 358 by their cell cycle scores.

FIG. 9B is a heatmap of core promoter activities in different cell cycle phases (Color bar indicates housekeeping (blue) vs developmental (red) promoters). Core promoter activities have been normalized within each cell cycle phase to highlight the differences between housekeeping and developmental promoters.

FIG. 9C is a UMAP embedding of K562 cells with high proliferation (CD34+/CD38− and CD24+362) and undifferentiated substates.

FIG. 9D is a heatmap showing hierarchical clustering showing two clusters (“up” and “down”) based on expression patterns in the three substates. The promoter activities are plotted as their z-score from the average across cell 365 states to highlight the difference between cell states.

FIG. 9E is a graph of the proportion of promoters in the up and down clusters that contain the indicated core promoter motif. Significant p-values from a two-sided Fisher's exact test are shown.

FIG. 10A is a schematic illustration showing Gnb3 promoter library constructs. In addition to the cBC and rBC barcodes, the Gnb3 promoter library contains an additional cassette in which the constitutive U6 promoter expresses a second copy of the cBC with a capture sequence for isolating these transcripts on gel beads.

FIG. 10B is a set of schematic illustrations of two different types of transcripts produced from the Gnb3 promoter library to measure promoter expression and detect unexpressed promoters respectively. The two types of transcripts originating from the same cell share the same 10× cell barcodes.

FIG. 10C is a schematic illustration of the experimental workflow for scMPRA in ex vivo mouse retinas.

FIG. 10D is a UMAP of all cells measured in scMPRA with four major cell types identified.

FIG. 10E is a graph that shows for each Gnb3 variant in the library, the proportion of cells that contain barcoded poly(A) transcripts out of all the cells that contained the variant was determined.

FIG. 10F is a set of graphs showing reproducibility of promoter activities between biological replicates in each of the four major cell types.

FIG. 11A is a graph of the expression of the wild-type Gnb3 promoter in scMPRA reflecting endogenous expression levels of Gnb3 in the respective cell types. The solid line represents the best fit linear regression.

FIG. 11B is a graph of the expression of the entire Gnb3 library (n=115 variants) in different cell types that also follows endogenous Gnb3 expression (****: p-value <0.0001, two-sided Mann-Whitney U test).

FIG. 11C is a graph showing that scMPRA recapitulates the effects of a known Gnb3 variant, where the CRX3Q50/CRX5Q50 variant reduces expression in bipolar cells specifically (*: p-value <0.05, two-sided Welch's t-test). All expression values are plotted as the mean of two biological replicates.

FIG. 12A is a schematic illustration of the Gnb3 promoter showing the location of the five CRX binding sites and the E-box.

FIG. 12B is a plot of the effects of individual and pairwise deletions of CRX binding sites.

FIG. 12C is a plot of the effects of individual and pairwise mutations of CRX K50 binding sites to Q50 binding sites.

FIG. 12D is a plot of the effects of changing CRX binding site affinities.

FIG. 12E is a plot of the effects of saturation mutagenesis of the E-box.

FIG. 12F is a plot of the effects of shuffle mutants in conserved regions of the Gnb3 promoter. Each region was split into 5 bp windows and the nucleotides in each window were shuffled. Labels above the heatmap indicate locations where the mutations impact CRX or E-box binding sites. All plots show log 2 fold changes of the mutant relative to WT Gnb3 expression in that cell type. Stars above the plot indicate a significant cell-type specific effect by one-way ANOVA.

FIG. 13A is a UMAP from a total of 3112 cells that shows that 97% could be unambiguously assigned to one of the two cell types.

FIG. 13B is a set of UMAPs that shows that 97% of the cells could be assigned to one of the two cell types.

FIG. 13C is a histogram wherein the number of cBC-rBC pairs in each individual cell was tabulated, which found that the median per cell was 341 in HEK293 cells.

FIG. 13D is a histogram wherein the number of cBC-rBC pairs in each individual cell was tabulated, which found that the median per cell was 164 in K562 cells.

FIG. 13E is a histogram that shows, on average, 10 rBCs per promoter were detected in individual HEK293 cells.

FIG. 13F is a histogram that shows, on average, 2 rBCs per promoter were detected in individual K562 cells.

FIG. 13G is a plot that shows that in K562 cells, correlation drops with the UMI-corrected single-cell measurements compared to the uncorrected correlation described in FIGS. 8F and G.

FIG. 13H is a plot that shows that in a HEK293, correlation drops with the UMI-corrected single-cell measurements compared to the uncorrected correlation described in FIGS. 8F and G.

FIG. 13I is a set of two plots that shows that the quality of measurements between housekeeping and developmental promoters does not play a significant role.

FIG. 13J is a set of two plots that shows that the quality of measurements between housekeeping and developmental promoters does not play a significant role.

FIG. 14A is a plot that shows measurements of each library member was highly correlated between replicates and agreed well with independent bulk measurements.

FIG. 14B is another plot that shows measurements of each library member was highly correlated between replicates and agreed well with independent bulk measurements.

FIG. 14C is a set of plots wherein core promoters with different expression dynamics through the cell cycle were identified. It was found that the core promoter of UBA52 remains highly expressed in the S phase, whereas the core promoter of CXCL10 is lowly expressed throughout.

FIG. 14D is a plot to identify sub-states in the single-cell transcriptome data; the cell cycle effects were first regressed out and it was confirmed that the single cell transcriptome data no longer clustered by cell cycle phase.

FIG. 15A is a plot that shows the scRNA-seq data showed that rod photoreceptors (87.3%), bipolar cells (3.5%), interneurons (i.e. amacrine cells) (5.2%), and Müller glia cells (3.9%) were recovered.

FIG. 15B is a pie chart that shows rod photoreceptors (87.3%), bipolar cells (3.5%), interneurons (i.e. amacrine cells) (5.2%), and Müller glia cells (3.9%) were recovered with scRNA-seq.

FIG. 15C is a set of graphs that shows transfection was linearly related to the strength of the promoter, with stronger promoters expressing in a larger fraction of cells.

FIG. 15D is a graph that shows the minimum number of cells required for reproducible measurements (Spearman's ρ>0.75) of mean reporter gene levels is 75 cells.

Those of skill in the art will understand that the drawings, described below, are for illustrative purposes only. The drawings are not intended to limit the scope of the present teachings in any way.

### DETAILED DESCRIPTION

The present disclosure is based, at least in part, on the discovery that Massively Parallel Reporter Assays (MPRAs) can be designed to quantify activity of regulatory elements in single cells of a heterogeneous population (scMPRA). As shown herein, scMPRA is a scalable technology that can be used to assay cis-regulatory sequence function in diverse cell types.

Described herein is a method to perform Massively Parallel Reporter Gene Assays (MPRA) in single cells (scMPRA). In scMPRA, a library of reporter genes is introduced into a complex mixture of cells and the user gets back two types of information: the single-cell RNA-seq profile of each individual cell and the activities of the reporter genes in each individual cell. With these two types of data, an investigator can determine how a library of gene regulatory elements behaves across multiple cell types without having to separate the cell types.

MPRAs are the workhorse technology for investigators and companies interested in identifying gene regulatory elements that are active in specific cell types. MPRAs provide power by allowing investigators to assay many regulatory elements in parallel. The primary limitation of MPRAs is that they can only be performed in one cell type at a time, or as bulk assays that represent the average of many cell types.

The scMPRA method disclosed herein allows investigators to assay the same MPRA library across multiple cell types simultaneously. The disclosed scMPRA method includes two separate barcodes onto MPRA library vectors. These barcodes get incorporated into the mRNA produced from each reporter gene in the library. One barcode specifies the identity of the cisregulatory element (CRE) that drives the reporter gene (cBC or BC1). A second highly diverse random barcode (rBC or BC2) is also incorporated into the libraries.

After introduction into cells, individual cells are prepared for single-cell sequencing. The mRNAs produced from individual copies of each CRE in each cell are tabulated from the cBC-rBC pairs in each individual cell, and the cell type of each cell is identified from the scRNAseq profile of cellular mRNAs. In this way, the activities of a diverse set of CREs are measured in a diverse set of cell types in a single experiment.

As shown herein, the use of scMPRA has been successfully piloted and used to discover gene promoters with differential activity between two commonly used cell lines, HEK293 and K562. scMPRA has also been used to identify promoters that are active in different subpopulations of K562 and in different phases of the cell cycle. The ability of scMPRA to identify regulatory elements that are active in different cell types of the nervous system is currently being tested.

One use of this technology will be for the discovery of natural and synthetic regulatory elements that drive potent levels of gene expression in specific cell types. The advent of CRISPR technology has led to a resurgence of interest in gene therapy. However, a limitation of gene therapy is figuring out how to express the therapeutic in only the desired cell type. One major avenue of attack on this problem is to identify natural or synthetic elements that are active only in specific cell types to act as drivers on gene therapy vectors. scMPRA will allow investigators and companies to screen large libraries of potential regulatory activities in multiple cell types simultaneously.

scMPRA addresses both the scale and cell-type specificity of assays for CREs with defined properties. scMPRA synergizes with advances in DNA delivery by AAV viral vectors. With AAV, reporter genes can be delivered in vivo to complex mixtures of cells. However, because in vivo delivery involves multiple cell types, the resulting reporter gene activity is an average over many (often unknown) cell types. With scMPRA, the reporter genes are quantified in each distinct cell type simultaneously. In addition to its use in defining cell type specific regulatory elements, scMPRA can also be used to identify gene therapy vectors and variants with new cell tropisms.

Because scMPRA provides the activities of CREs across multiple individual cells, both the average activity of each element and the variance of each element across cells is measured. Thus, scMPRA can be used to identify regulatory elements with higher or lower cell-to-cell variability, which could help improve the efficacy of cellular reprogramming protocols that are currently plagued by non-uniform expression of lineage-determining transcription factors.

Bar Codes

The present disclosure provides for a plurality of expression vectors comprising at least one bar code. As described herein, scMPRA may use a two-level barcoding scheme that allows for measuring the copy number of all the reporter genes in a single cell from mRNA alone.

A specific barcode marks each cis-regulatory sequence of interest (CRS barcode, “cBC” or BC1). In some embodiments, multiple barcodes (e.g., cBCs or BC1s) may be associated with a CRS to provide redundancy in the measurements. For example, each CRS may be associated with at least about 2 barcodes, at least about 3 barcodes, at least about 4 barcodes, at least about barcodes, at least about 6 barcodes, at least about 7 barcodes, at least about 8 barcodes, at least about 9 barcodes, or at least about 10 barcodes.

A second random barcode (rBC or BC2) acts as a proxy for DNA copy number of reporter genes in single cells. The rBC is complex enough to ensure that the probability of the same cBC-rBC appearing in the same cell more than once is vanishingly small. In this regime, the number of different cBCrBC pairs in a single cell becomes an effective proxy for the copy number of a CRS in that cell.

The length of the random barcode is not constrained to any specific length, provided the complexity of the random barcode is sufficient to determine the copy number of a CRS in a cell. By way of non-limiting example, the random barcode may be at least about 25 nucleotides long.

Even if a cell carries reporter genes for multiple different CRS, and each of those reporter genes is at a different copy number, it is still possible to normalize each reporter gene in each individual cell to its plasmid copy number. With this barcoding scheme, the activity of many CRSs with different input abundances can be measured in single cells.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasm id or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amounts of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at ° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/1). Furthermore, the Tₘ of a DNA: DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Screening

Also Provided are Screening Methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to nucleic acid vectors, reporter constructs, bar code constructs, or cells. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Overview of Single Cell Massively Parallel Reporter Assay (scMPRA)

To develop the scMPRA method disclosed herein the following experiments were conducted.

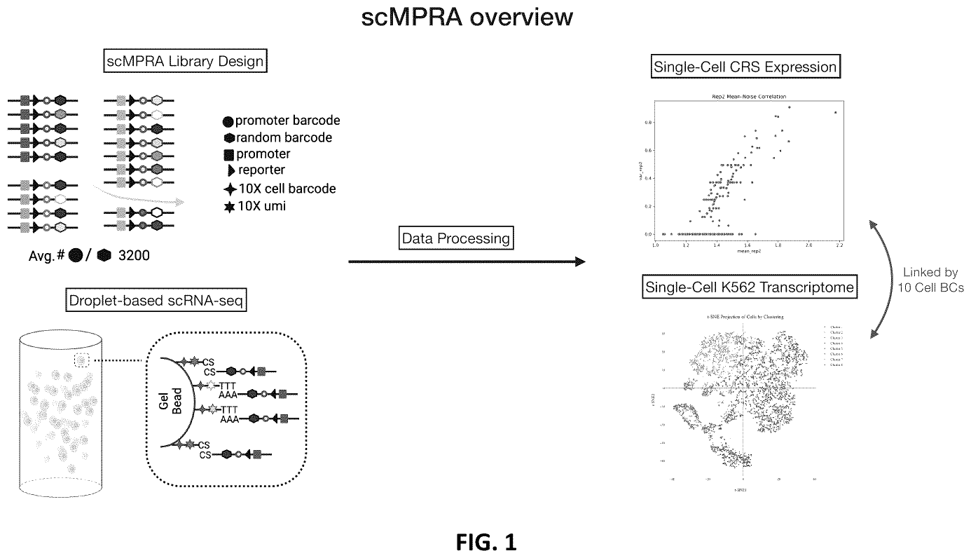

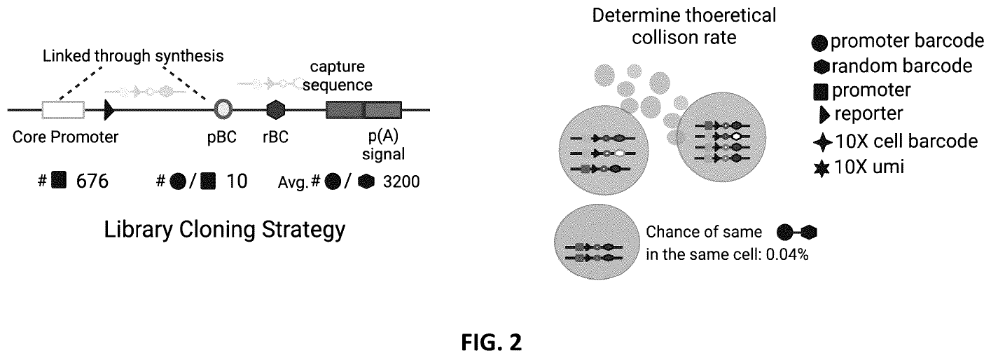

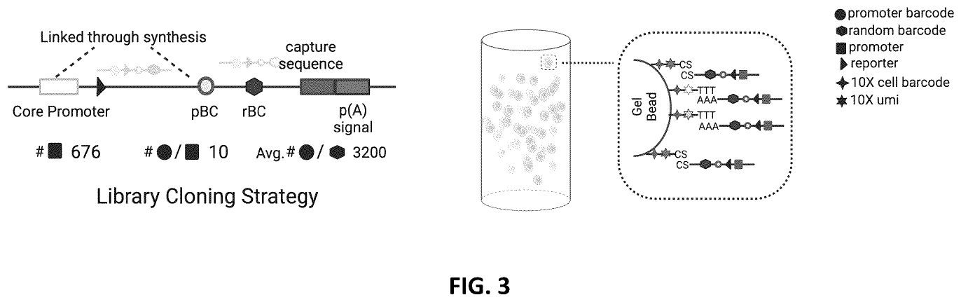

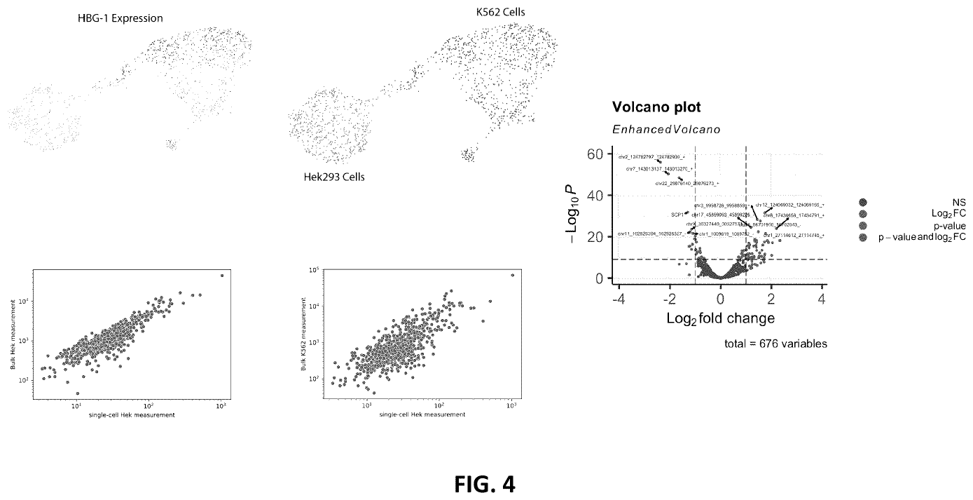

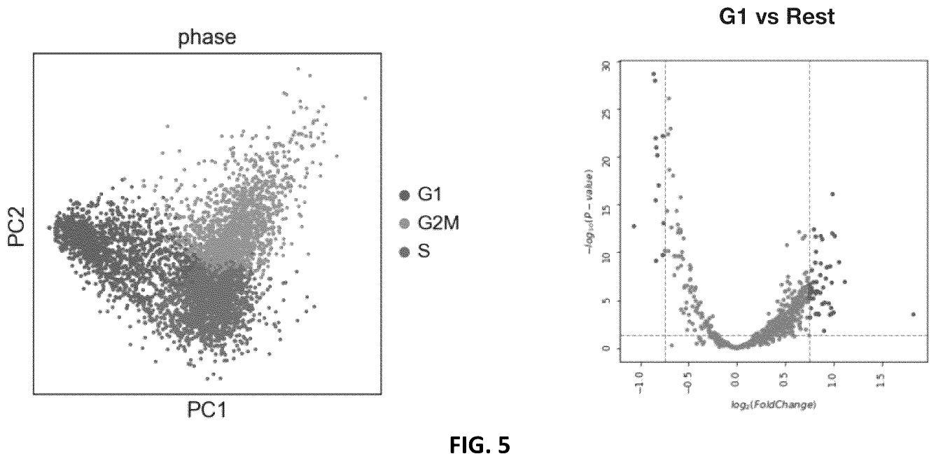

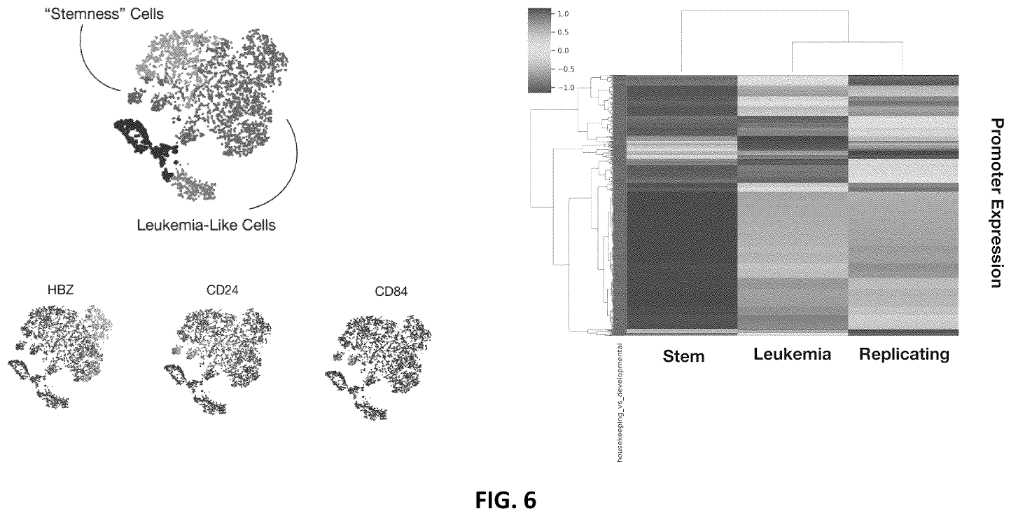

This example describes the design and implementation of Massively Parallel Reporter Assays to quantify activity of regulatory elements in single cells of a heterogeneous population (scMPRA). An overview schematic of scMPRA is shown in FIG. 1. The complex barcoding strategy enables input plasmid normalization (FIG. 2), and double-capturing sequences allow for efficient capturing (FIG. 3). The assay is able to detect cell-type specific activity in mixed cell populations (FIG. 4), cell-cycle specific CRS expression can be identified (FIG. 5), and CRS-specific expression in rare populations of a cancer cell line can be identified (FIG. 6).

### Example 2: A Single-Cell Massively Parallel Reporter Assay Detects the Effect of Cell Type Specific Cis-Regulatory Activity

This example describes the development of a single cell Massively Parallel Report Assay (scMPRA) and its use in measuring activity of libraries of cis-regulatory sequences (CRSs) across multiple cell types simultaneously.

### Abstract

Massively parallel reporter gene assays are important tools in regulatory genomics, but cannot be used to identify cell-type specific regulatory elements without performing assays serially across different cell types. To address this problem, a single-cell massively parallel reporter assay (scMPRA) was developed to measure the activity of libraries of cis-regulatory sequences (CRSs) across multiple cell-types simultaneously. A library of core promoters in a mixture of HEK293 and K562 cells was assayed and showed that scMPRA is a reproducible, highly parallel, single-cell reporter gene assay that detects cell-type specific cis-regulatory activity. A library of promoter variants was then measured across multiple cell types in live mouse retinas and showed that subtle genetic variants can produce cell-type specific effects on cis-regulatory activity. It is anticipated that scMPRA will be widely applicable for studying the role of CRSs across diverse cell types.

### Results

scMPRA Enables Single-Cell Measurement of CRS Activity

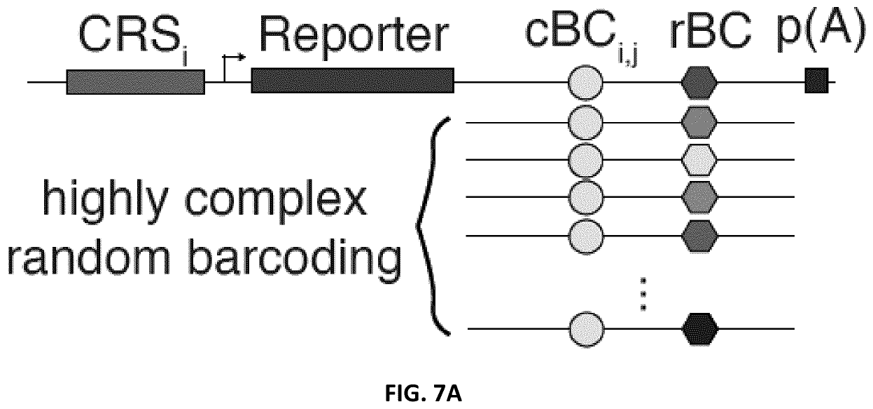

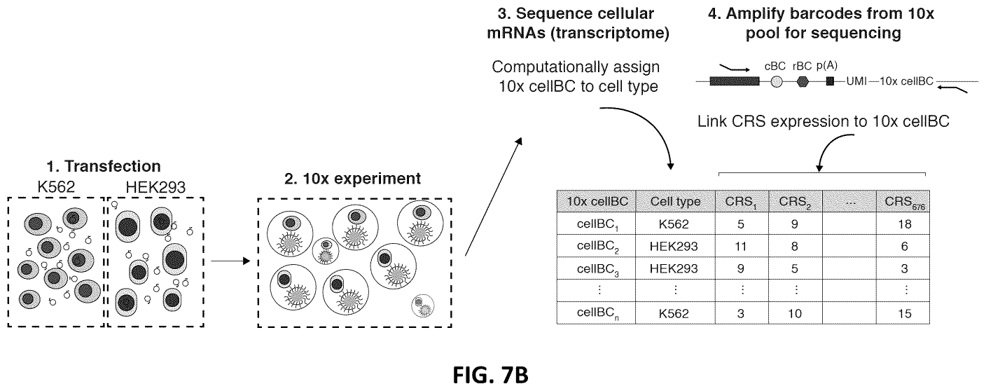

scMPRA, a procedure that combines single-cell RNA sequencing with MPRA was developed. scMPRA simultaneously measures the activities of reporter genes in single cells and the identities of those cells using their single-cell transcriptomes. One component of scMPRA is a two-level barcoding scheme that enables measuring the copy number of all reporter genes present in a single cell from mRNA alone. A specific barcode marks each CRS of interest (CRS barcode, “cBC”), and a second random barcode (rBC) acts as a proxy for DNA copy number of reporter genes in single cells (FIG. 7A). The critical aspect of the rBC is that it is complex enough to ensure that the probability of the same cBC-rBC appearing in the same cell more than once is vanishingly small. In this regime, the number of different cBC-rBC pairs in a single cell becomes an effective proxy for the copy number of a CRS in that cell. Even if a cell carries reporter genes for multiple different CRSs, and each of those reporter genes is at a different copy number, each reporter gene in each individual cell can still be normalized to its plasmid copy number. With this barcoding scheme, one can measure the activity of many CRSs with different input abundances at single-cell resolution, which allows one to measure the activity of CRSs simultaneously across different populations of cells.

As a proof of principle, scMPRA was first used to test whether different classes of core promoters show different activities in different cell types. Core promoters are the non-coding sequences that surround transcription start sites, where general cofactors interact with RNA polymerase II. Core promoters are divided into different classes by the functions of their host genes (housekeeping vs developmental), as well as by the sequence motifs they contain (TATA-box, downstream promoter element (DPE), and CpG islands). 676 core promoters that we previously tested were selected and then cloned into a double-barcoded MPRA library. Given the complexity of the library (>1×10⁷ unique cBC-rBC pairs), it was calculated that the probability of plasm ids with the same cBC-rBC pair occurring in the same cell is less than 0.01 with our transfection protocols (Methods). Given this low likelihood, the number of rBC per cBC in a cell represents the copy number of a CRS in that cell. Knowing the copy number of CRSs in single cells allows one to normalize reporter gene expression from each CRS to its copy number in individual cells.

A cell mixing experiment was performed to test whether scMPRA could measure cell type specific expression of reporter genes. K562 (chronic myelogenous leukemia) and HEK293 (human embryonic kidney) cells were transfected, and scMPRA was performed on a 1:1 mixture of those cell lines (FIG. 7B). The mRNA from single cells was captured, converted to cDNA, and sequenced. The resulting cBC-rBC abundances and transcriptome of each single cell are linked by their shared 10× cell barcode.

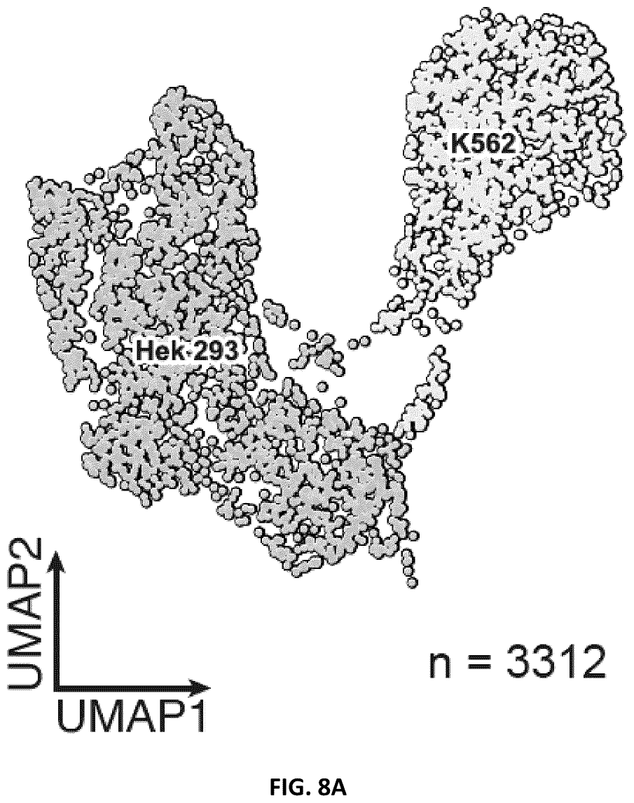

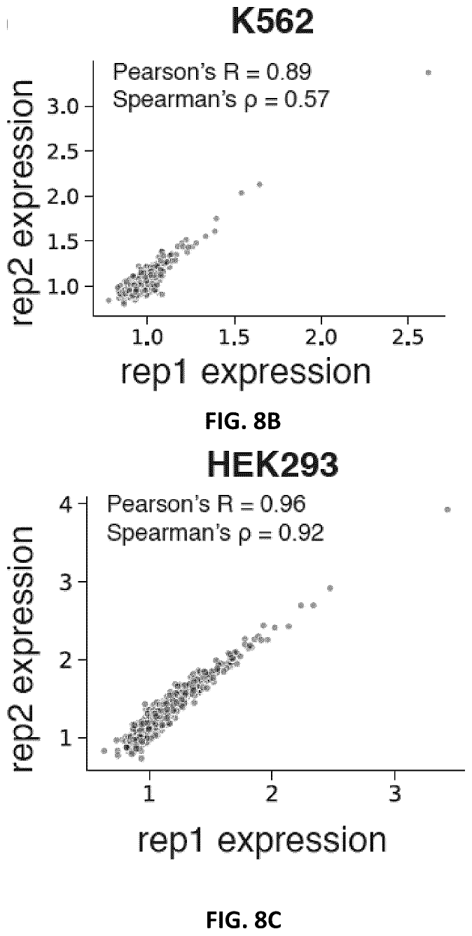

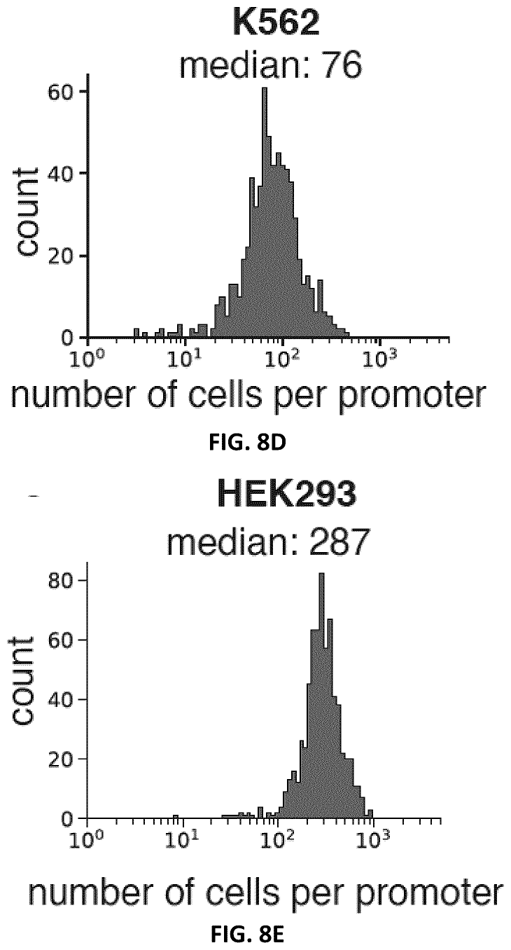

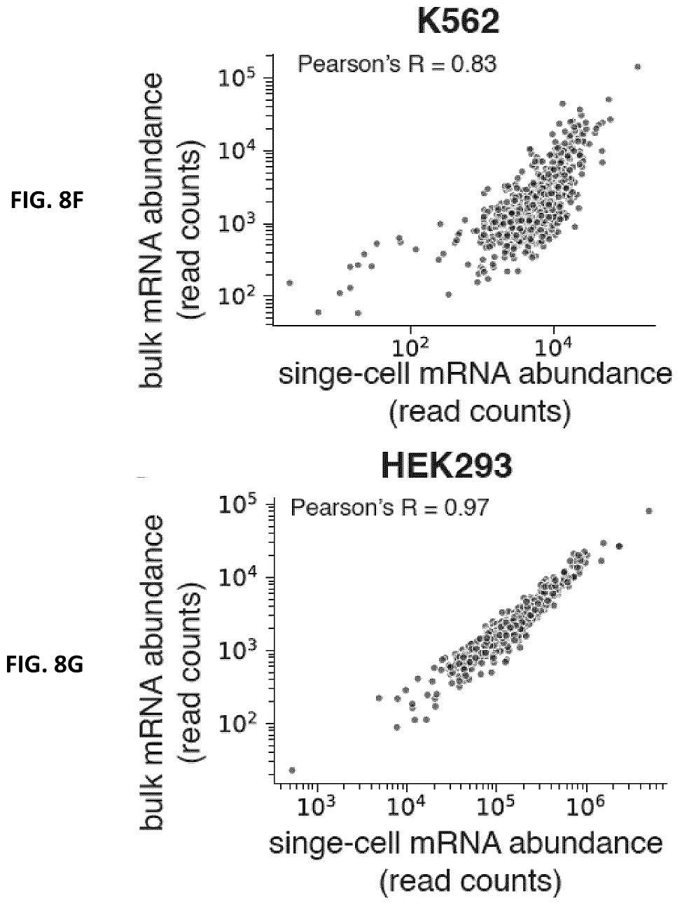

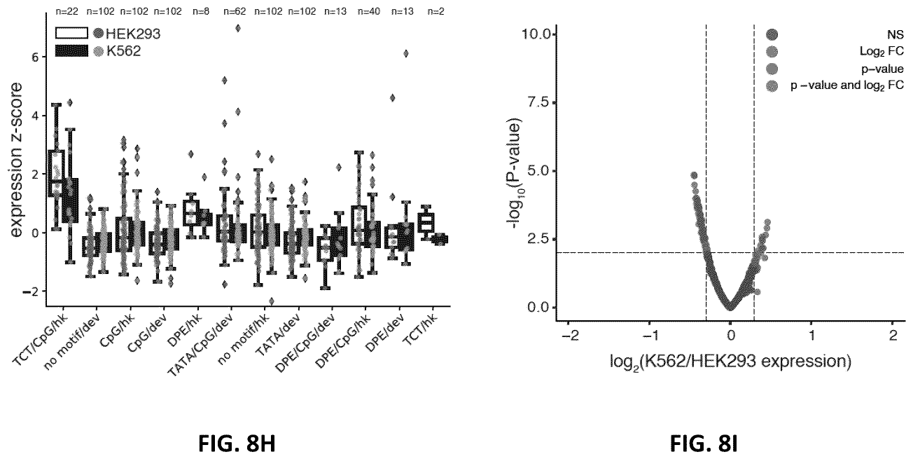

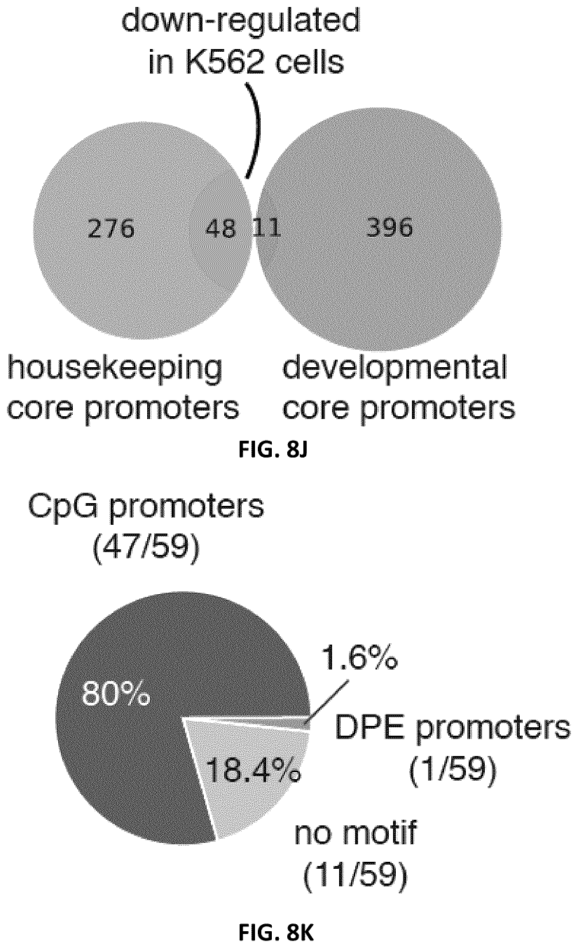

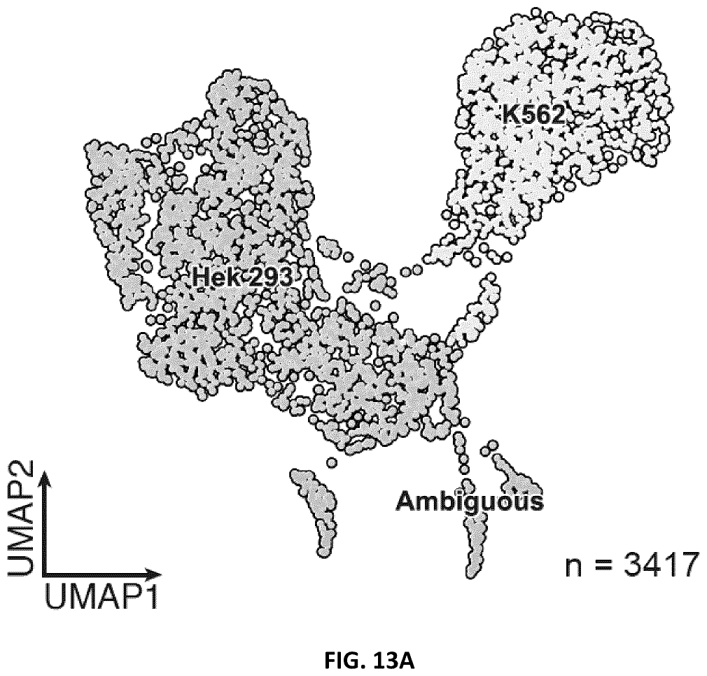

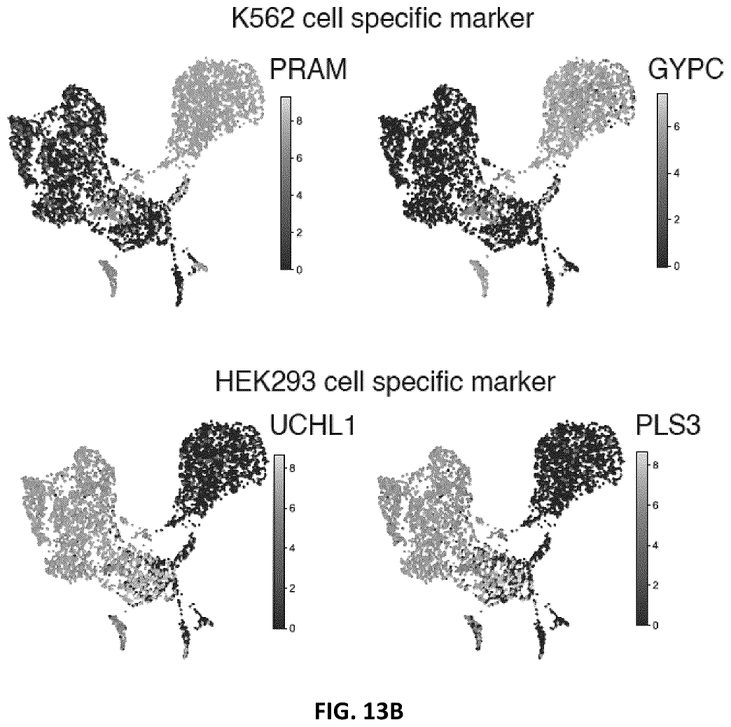

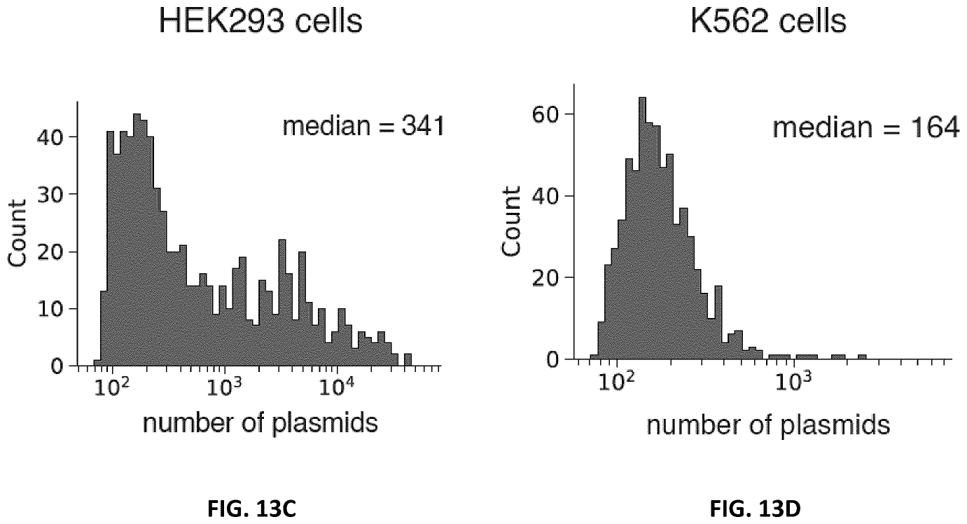

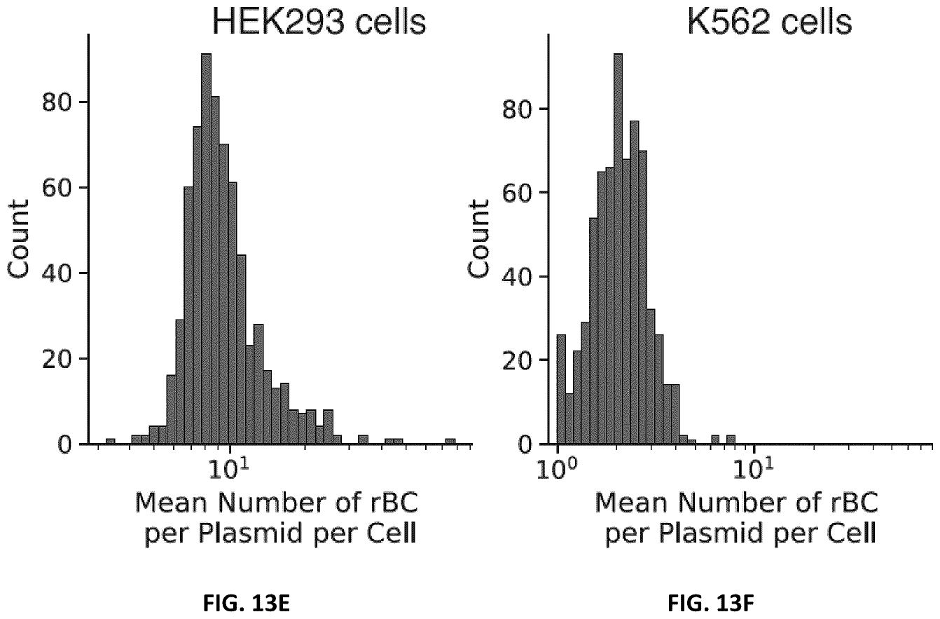

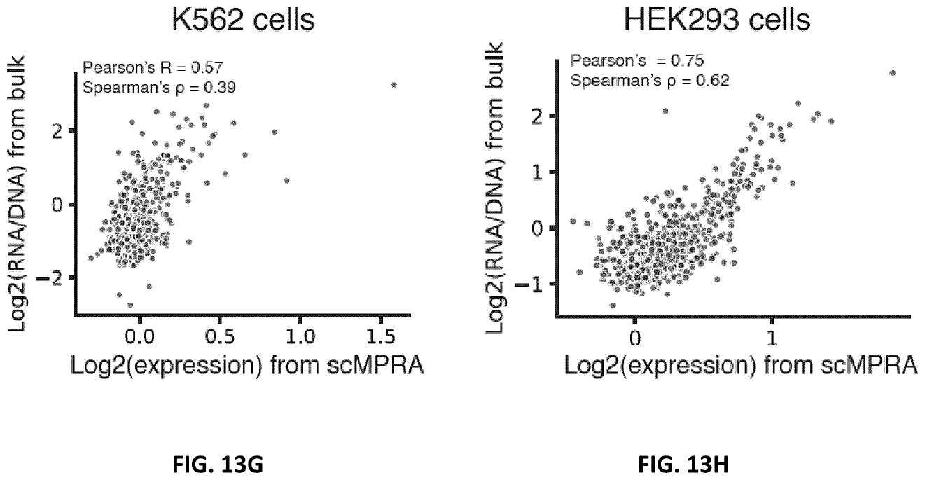

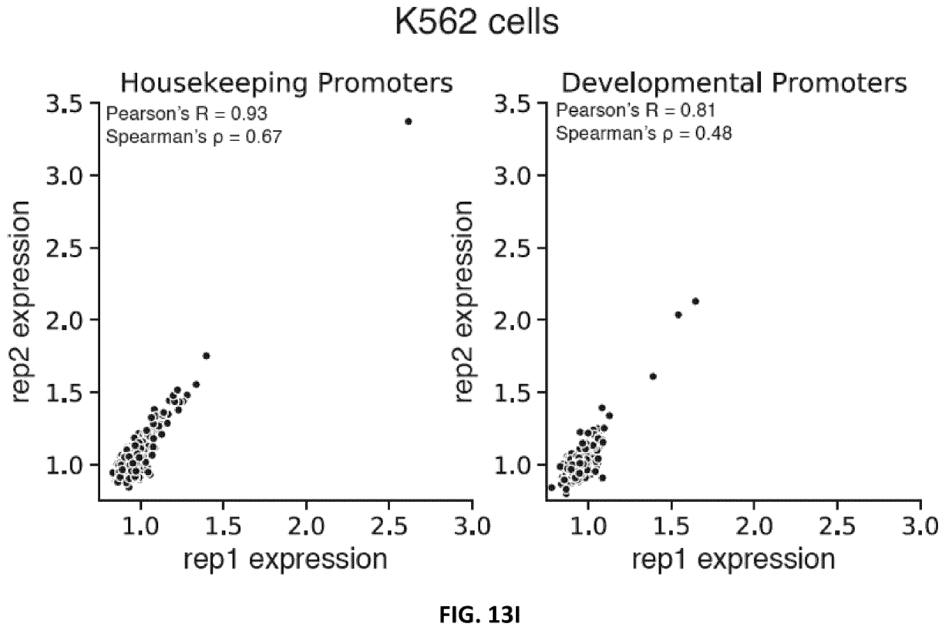

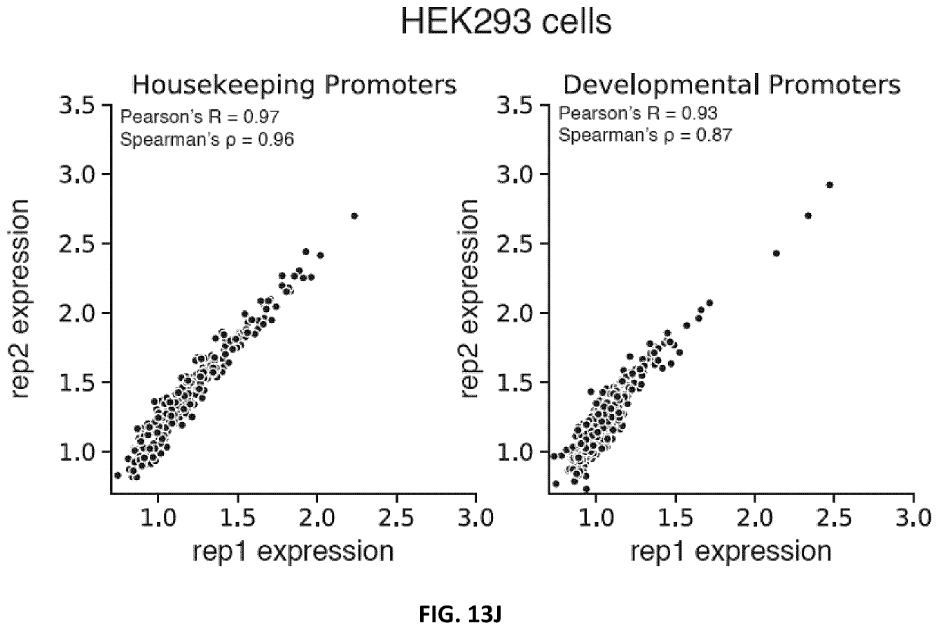

A total of 3112 cells (97%) were recovered that could be unambiguously assigned to one of the two cell types (FIG. 8A, FIGS. 13A and B) and the mean expression of each core promoter in the library in each cell type was computed (Methods). The measurements were reproducible in both cell types (K562: Pearson's R=0.89, Spearman's p=0.57, HEK293: Pearson's R=0.96, Spearman's p=0.92) (FIGS. 8B and C), and measurements were obtained for 99.5% of core promoters in K562 cells and 100% in HEK293 cells, highlighting the efficiency of scMPRA. The median number of cells in which each core promoter was measured was 76 for K562 cells and 287 for HEK293 cells (FIGS. 8D and 8E). The number of cBC-rBC pairs in each individual cell was tabulated and it was found that the median per cell was 164 in K562 cells and 341 in HEK293 cells (FIGS. 13C and 13D). On average 10 rBCs were detected per promoter in individual HEK293 cells and 2 rBCs per promoter in K562 cells (FIGS. 13E and 13F). To validate the scMPRA measurements, we conducted bulk MPRA of the core promoter library was conducted in the two cell types separately. Bulk MPRA measurements are not corrected for PCR amplification biases with UMIs, and it was found that the bulk measurements correlate well with aggregated single-cell measurements without UMI correction (FIGS. 8F and 8G). That correlation drops with the UMI-corrected single-cell measurements (FIGS. 13G and 13H), which suggests that bulk measurements may suffer from over counting because of uneven amplification during PCR.

scMPRA Detects Cell Type Specific CRS Activity

It was asked whether the data allowed one to detect core promoters with differential activity between K562 and HEK293 cells. While different classes of core promoters generally had similar activities in both cell lines (FIG. 8H), the differential analysis using DEseq226 identified a small number of promoters (11 out of 669) that are upregulated in K562 cells, and 59 promoters that are downregulated in K562 cells (adjusted p-value <0.01, log 2 fold change >0.3, FIG. 8I). Among the down-regulated promoters, 48 out of 59 core promoters belong to housekeeping genes (p=1.08×10⁻¹¹, FIG. 8J), and 46 out of 59 core promoters are CpG-island-containing core promoters (p=2.18×10⁻⁶, FIG. 8K). This result is not due to differences in the quality of measurements between housekeeping and developmental promoters (FIGS. 131 and 13J). These results demonstrate the ability of scMPRA to detect CRSs with cell-type specific activities.

scMPRA Detects Cell Sub-State Specific CRS Activity

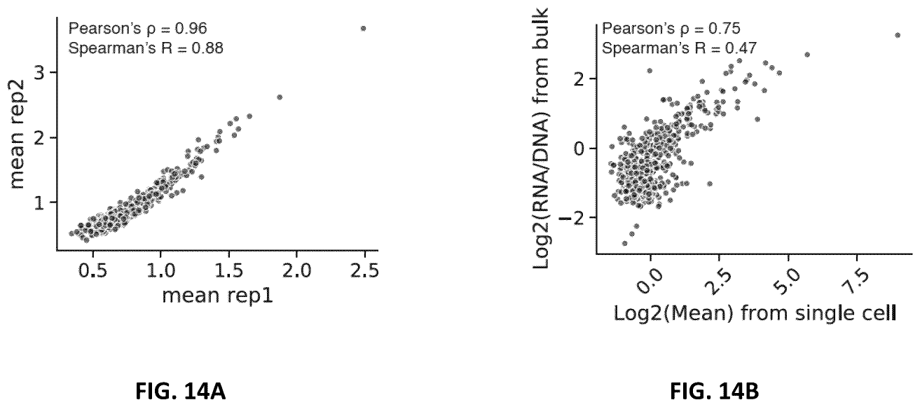

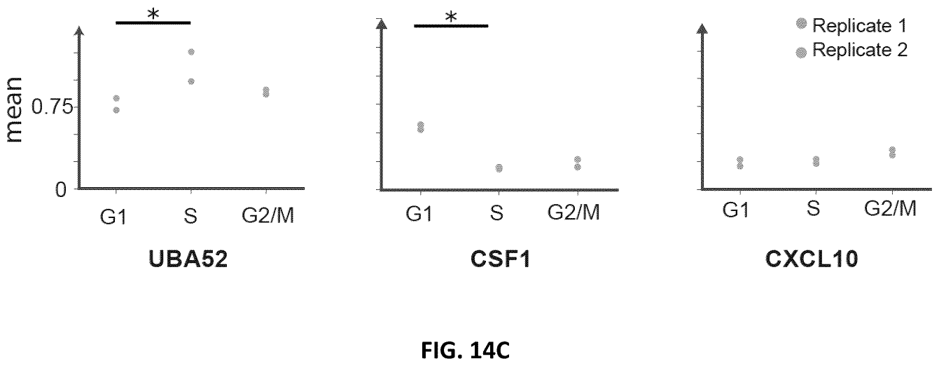

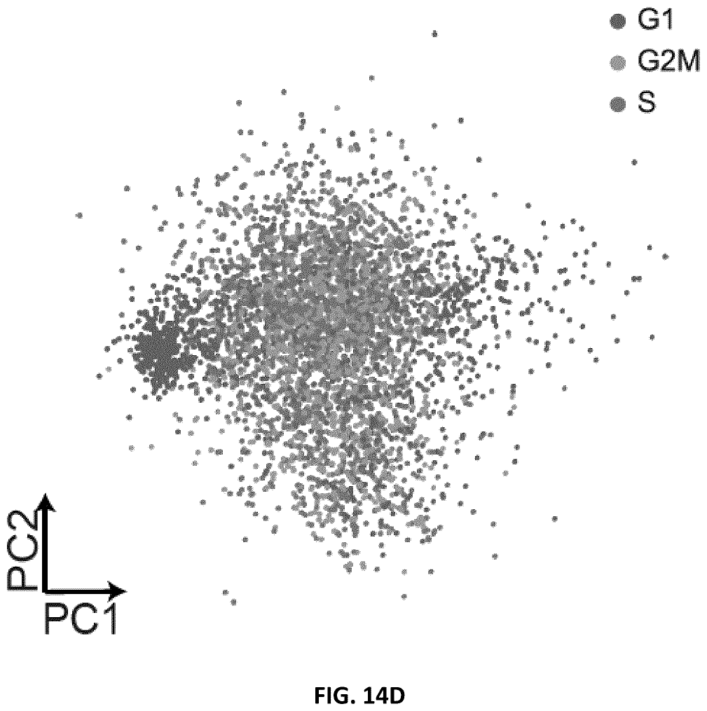

Single-cell studies have revealed heterogeneity in cell states even within isogenic cell types. Therefore, it was explored whether scMPRA can identify CRSs with cell-state specific activity. scMPRA was repeated on K562 cells alone and a total of 5141 cells from two biological replicates were obtained. Measurements of each library member were again highly correlated between replicates and agree well with independent bulk measurement (FIGS. 14A and 14B).

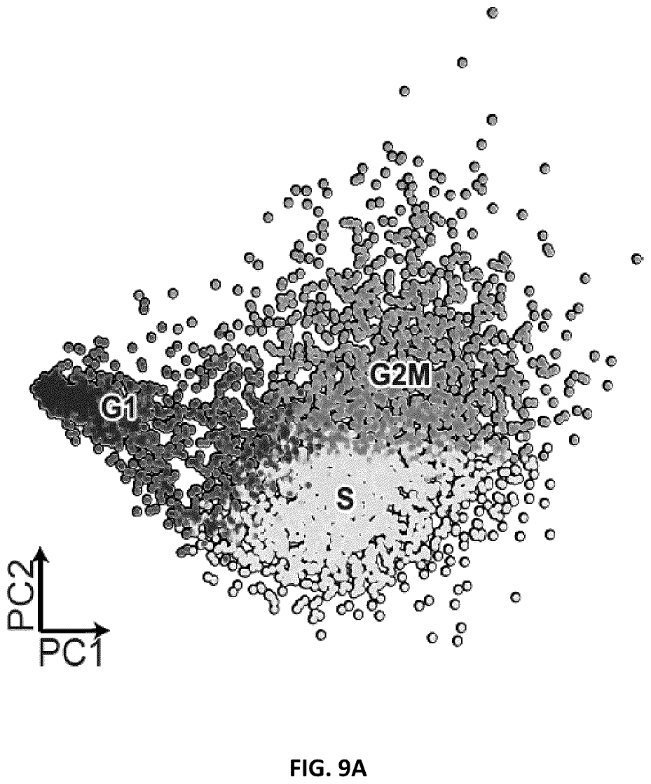

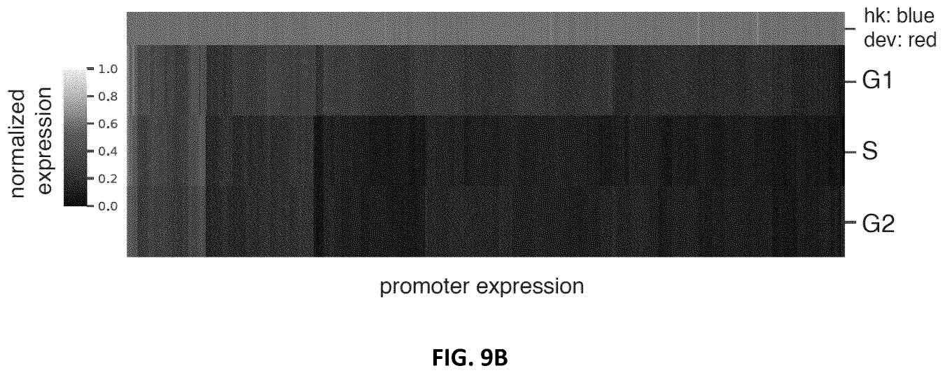

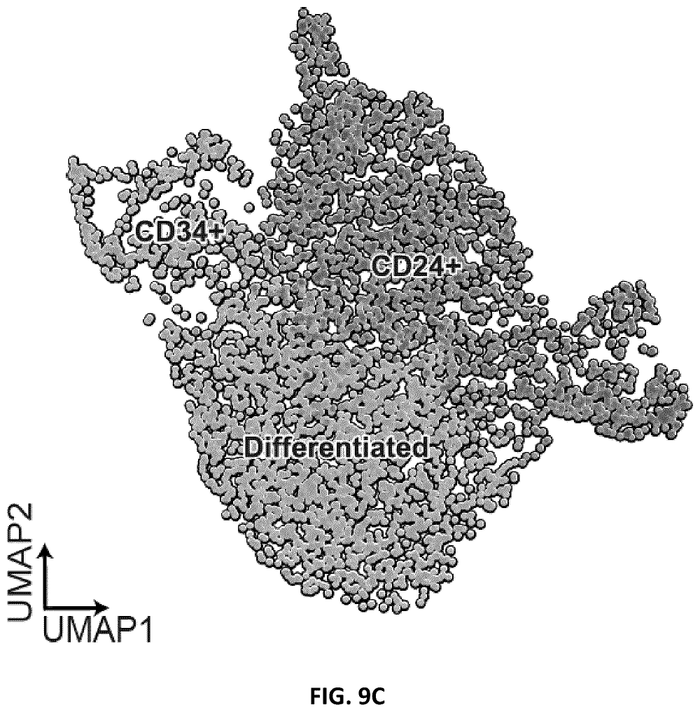

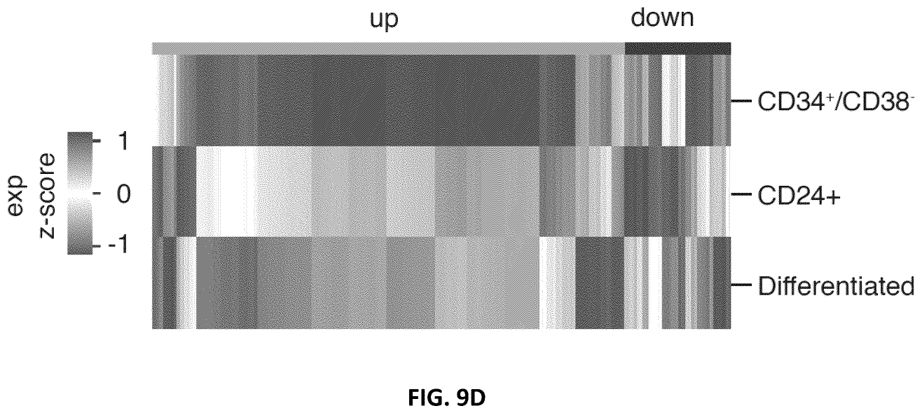

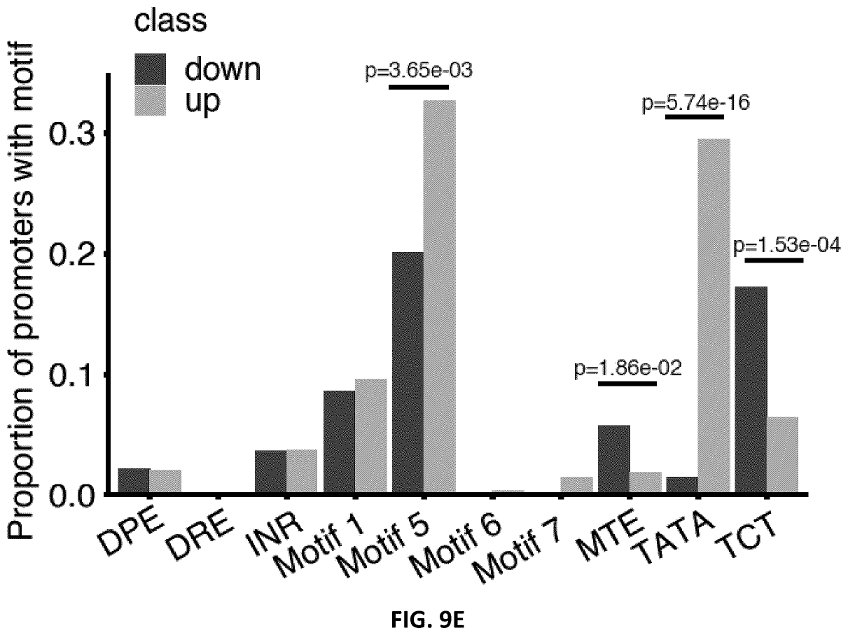

Because the phases of the cell cycle represent distinct cell-states, it was asked whether scMPRA could identify reporter genes with differential activity through the cell cycle. Cell cycle phases were assigned to each cell using their single cell transcriptome data (FIG. 9A) and the mean expression of each reporter gene in different cell cycle phases was calculated. It was found that most core promoters in the library are upregulated in the G1 phase of the cell cycle and that some housekeeping promoters are highly expressed through all cell cycle phases (FIG. 9B). We also identified core promoters with different expression dynamics through the cell cycle. For example, it was found that the core promoter of UBA52 remains highly expressed in the S phase, whereas the core promoter of CXCL10 is lowly expressed throughout (FIG. 14C). This analysis illustrates the ability of scMPRA to identify CRSs whose expression naturally fluctuates with cellular dynamics.

It was then asked whether scMPRA could detect reporter genes with activities that were specific to other cell-states in K562 cells, after normalizing for cell cycle effects. Two specific sub-states that have been reported and experimentally validated for high proliferation rates in K562 cells were focused on. The first is the CD34+/CD38− sub-state that has been identified as a leukemia stem cell subpopulation, and the second is the CD24+ sub-state that is linked to selective activation of proliferation genes by bromodomain transcription factors. To identify these sub-states in our single-cell transcriptome data, the cell cycle effects were first regressed out and it was confirmed that the single cell transcriptome data no longer clustered by cell cycle phase (FIG. 8D). Then clusters within K562 cells that have the CD34+/CD38− expression signature, or the CD24+ signature (FIG. 9C), were identified. Although the CD34+/CD38− cells represent only 9.3% of the cells, scMPRA revealed two distinct classes of core promoters that are upregulated and downregulated in these cells relative to the CD24+ and “differentiated” clusters (FIG. 9D). Conversely, the expression patterns of promoters are similar between the CD24+ and “differentiated” clusters (FIG. 9D). Motif analysis of the up/down regulated classes of promoters in CD34+/CD38− cells showed that different core promoter motifs are enriched in each class, with the TATA box and Motif 5 being enriched in the upregulated class and MTE and TCT motifs being enriched in downregulated class (FIG. 9E). This result suggests that differences in core promoter usage might be driving the differences between CD34+/CD38− and the other clusters. Because the TATA box is mostly found in developmental core promoters, the CD34+/CD38− subpopulation likely reflects the more “stem-like” cellular environment in these cells. The analysis highlights the ability of scMPRA to identify CRSs with differential activity in rare cell populations.

scMPRA is Reproducible and Accurate in Murine Retinas

To demonstrate that scMPRA is applicable in a complex tissue with multiple cell types, experiments were performed in explanted murine retinas. Intact retina from newborn mice can be cultured and transfected ex vivo. This system has been useful for bulk MPRA experiments, but the results from those experiments report the aggregate expression of library members across the cell types of the retina. Performing scMPRA in ex vivo retina provided a chance to assay an MPRA library in a living tissue with multiple cell types in their proper three-dimensional organization.

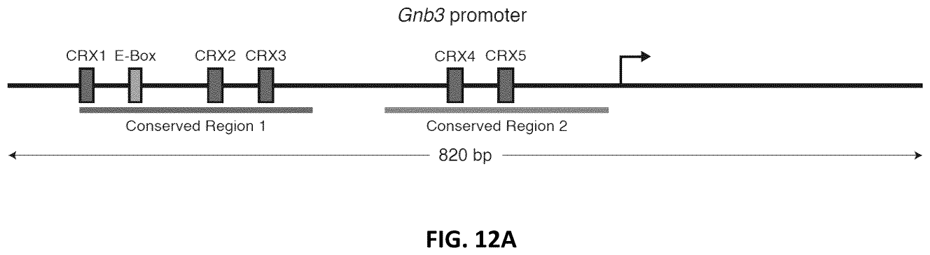

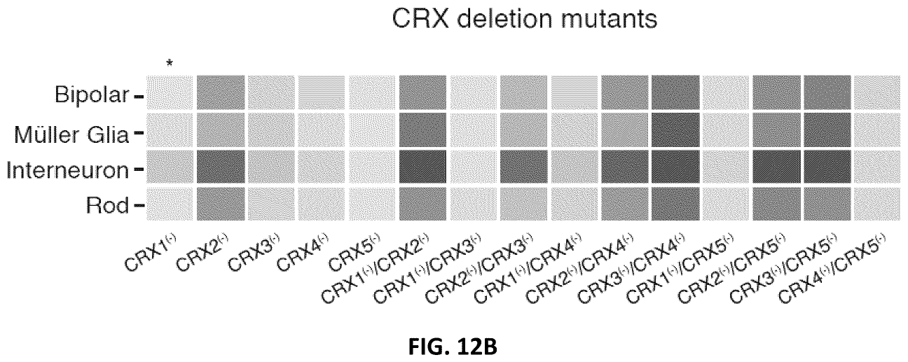

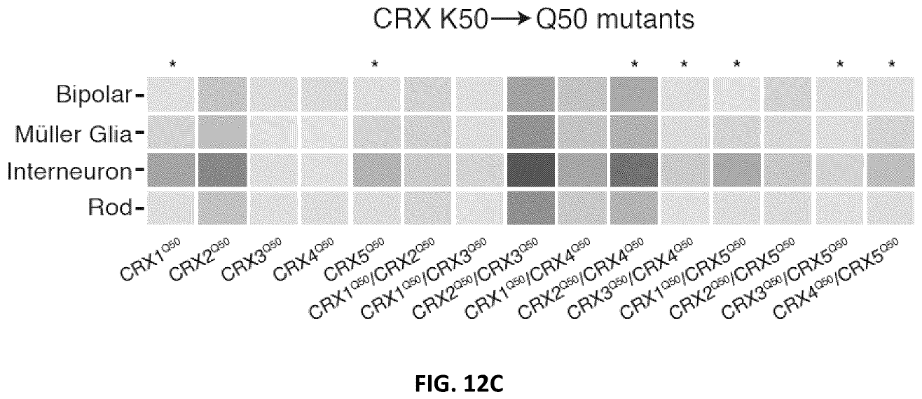

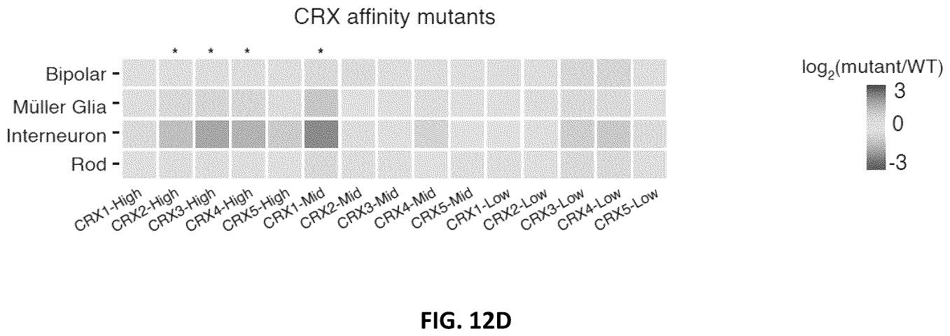

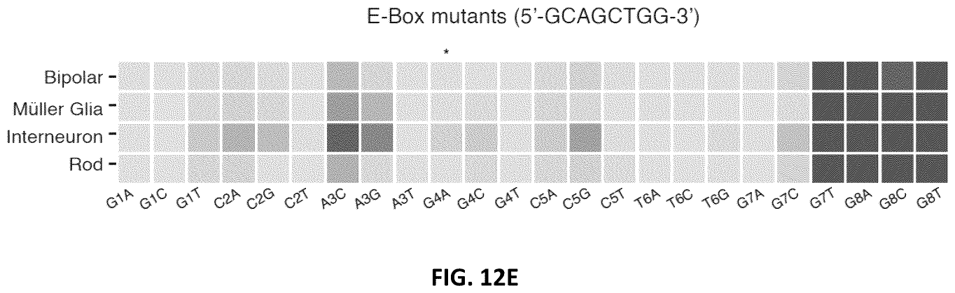

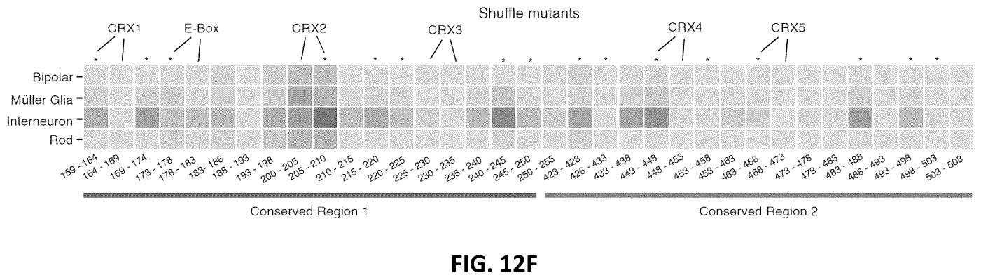

For this analysis, a library was designed consisting of two independently synthesized wild-type copies (SEQ ID NOS:125-126) and 113 variants (SEQ ID NOS:12-124) of the full-length Gnb3 promoter. The Gnb3 promoter was chosen because it has high activity in photoreceptors and bipolar cells, but lower expression in other interneurons (i.e. amacrine cells) and Müller glia cells. The library contains mutations in the known transcription factor binding sites (TFBSs) in the Gnb3 promoter as well as mutations that scan across two phylogenetically conserved regions of the promoter (FIG. 12). We constructed this library of Gnb3 promoter variants was constructed using the double barcoding strategy described above, with one modification that is now described.

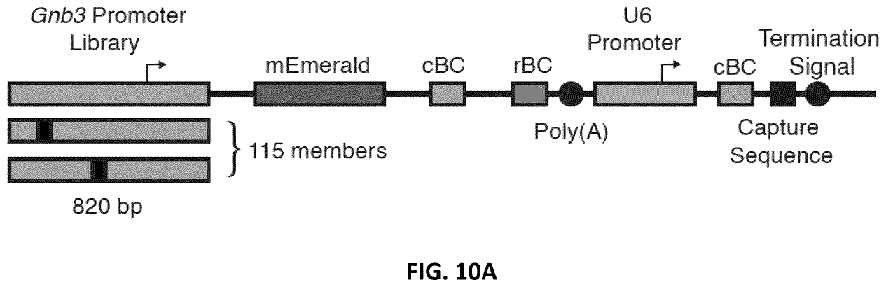

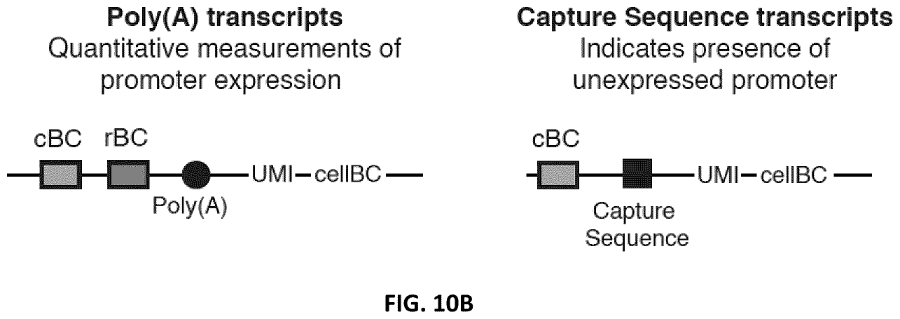

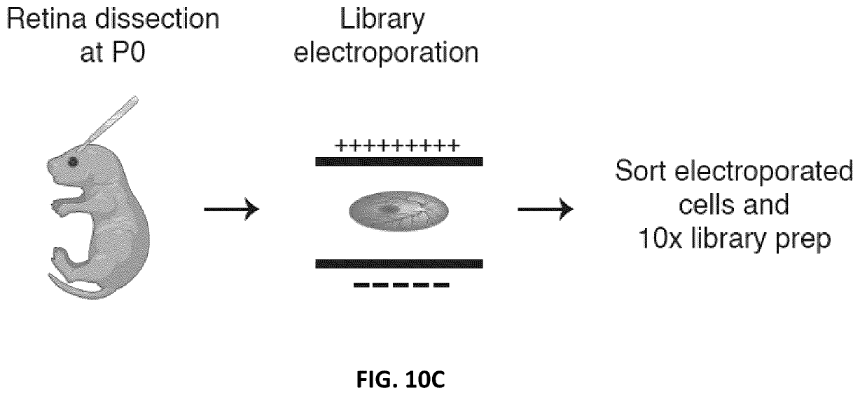

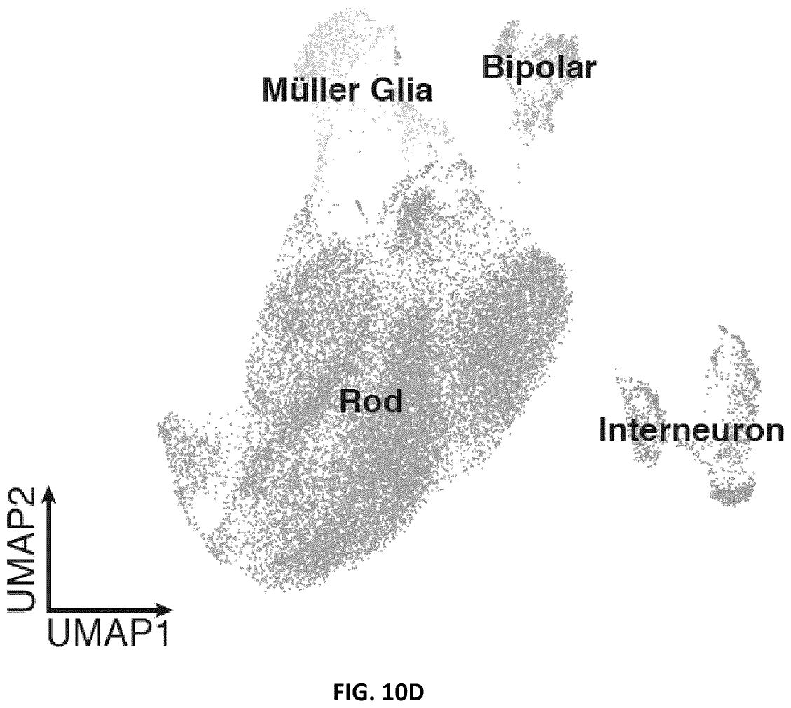

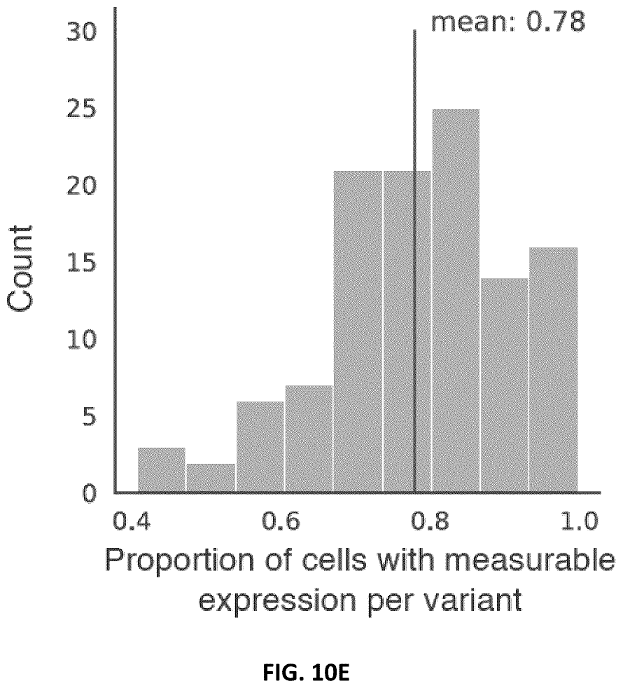

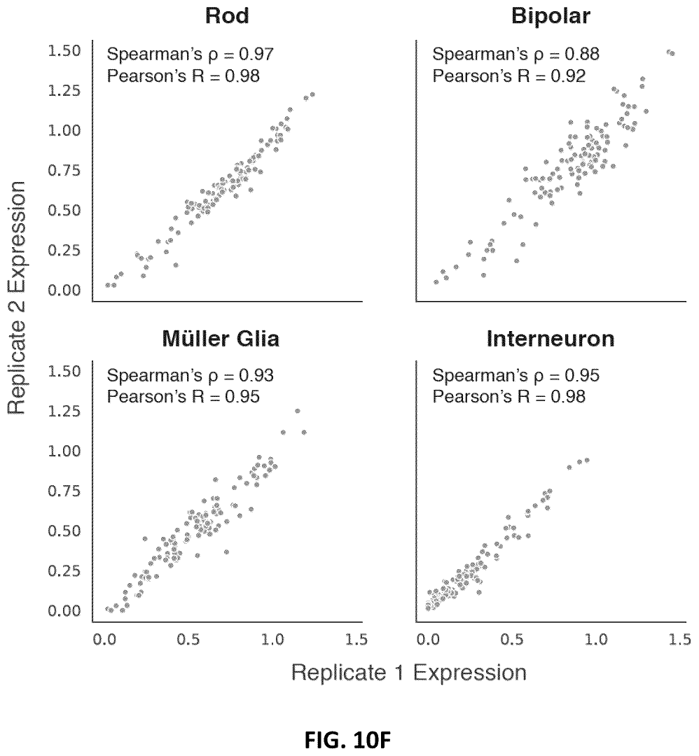

The inability of scMPRA to measure silent library members was addressed in the Gnb3 promoter library. In the first iteration of scMPRA, when a library member produces no mRNA barcodes its corresponding plasm id cannot be detected, and thus, a cell containing a silent plasmid is indistinguishable from a cell without a plasmid. To avoid this potential problem in our retina experiments, an additional cassette was included on the Gnb3 promoter library that allows one to detect the presence of plasm ids carrying silent promoter variants. A cassette was included in which the U6 promoter drives the expression of a second copy of the cBC coupled to the 10× Capture Sequence (FIG. 10A). The U6 promoter drives strong RNA Polymerase III-dependent transcription, and is independent of the activity of the Gnb3 promoter. While it is not expected to have interference between the pol III-dependent U6 promoter and the pol II-dependent Gnb3 promoter variants, to minimize this possibility the U6 cassette was put downstream of the Gnb3 variants and a polyA signal was placed between the cassettes. The Capture Sequence is a specific sequence that is typically used to identify gRNAs in Perturb-seq experiments, but it is used here to isolate U6 expressed cBCs (FIG. 10B). When a cell contains a U6 cBC without the corresponding Gnb3 promoter cBC, it indicates the presence of a silent library member. The individual cBCs associated with each library sequence are summarized in Table 1 below.

TABLE 1
Barcode Sequences of Gnb3
Promoter Library

| SEQ | |
|---|---|
| ID | BARCODE |
| NO | SEQUENCE |
| 12 | AACAACAC |
| 13 | AACAAGGT |
| 14 | AACACTGA |
| 15 | AACATTCC |
| 16 | AACCAGCC |
| 17 | AACCGTAA |
| 18 | AACCTCTT |
| 19 | AACGAGAA |
| 20 | AACGTCGC |
| 21 | AACTTGGA |
| 22 | AAGAATCG |
| 23 | AAGACCAA |
| 24 | AAGCGGTG |
| 25 | AAGCTACG |
| 26 | AAGGAGCT |
| 27 | AAGGTCAT |
| 28 | AATACCGC |
| 29 | AATAGTGG |
| 30 | AATCTCCA |
| 31 | AATGCCTT |
| 32 | ACAACTTC |
| 33 | ACAATAGC |
| 34 | ACACGCAA |
| 35 | ACAGGATT |
| 36 | ACCACAGT |
| 37 | ACCGACCT |
| 38 | ACCGTGTA |
| 39 | ACCTAGAT |
| 40 | ACCTTGCC |
| 41 | ACGAGTCC |
| 42 | ACGCATAA |
| 43 | ACGGACGA |
| 44 | ACGTATGG |
| 45 | ACGTTCAA |
| 46 | ACTAACCA |
| 47 | ACTATCTG |
| 48 | ACTCAGGT |
| 49 | ACTCCGAA |
| 50 | ACTTGTTG |
| 51 | AGAACAGA |
| 52 | AGAAGTAC |
| 53 | AGACGCTT |
| 54 | AGAGATGA |
| 55 | AGATGCGA |
| 56 | AGATTAGG |
| 57 | AGCCACTC |
| 58 | AGCCTGGT |
| 59 | AGCGAAGC |
| 60 | AGCTCTAA |
| 61 | AGGTAACG |
| 62 | AGGTGTCT |
| 63 | AGTACATC |
| 64 | AGTCCGTT |
| 65 | AGTGATTC |
| 66 | AGTTCGCA |
| 67 | ATAAGAGG |
| 68 | ATAAGCTC |
| 69 | ATATCACG |
| 70 | ATCCATGA |
| 71 | ATCGCCGT |
| 72 | ATCTAGCG |
| 73 | ATGACGGA |
| 74 | ATGCAACC |
| 75 | ATGGAATG |
| 76 | ATGTGCAG |
| 77 | ATTCCTAC |
| 78 | ATTGGTAG |
| 79 | CAACGCCA |
| 80 | CAAGAAGA |
| 81 | CAAGTCTG |
| 82 | CAATGGAC |
| 83 | CACACATC |
| 84 | CACATGCT |
| 85 | CACCTTAT |
| 86 | CACGGTAG |
| 87 | CAGAACCT |
| 88 | CAGAGGTT |
| 89 | CAGCCGAT |
| 90 | CAGTATAG |
| 91 | CATACTGT |
| 92 | CATCAAGT |
| 93 | CATCCACC |
| 94 | CATGTTCC |
| 95 | CATTGAGC |
| 96 | CCAACAAT |
| 97 | CCAAGCGT |
| 98 | CCAATTAC |
| 99 | CCACGACT |
| 100 | CCAGTGAA |
| 101 | CCATTGTC |
| 102 | CCGATCAG |
| 103 | CCGCATGT |
| 104 | CCGGTCTT |
| 105 | CCTACTCC |
| 106 | CCTCATAC |
| 107 | CCTCCTTG |
| 108 | CCTGAGTT |
| 109 | CCTTAATG |
| 110 | CCTTCGGA |
| 111 | CGAATATC |
| 112 | CGACAACG |
| 113 | CGAGAGCA |
| 114 | CGCCAGTA |
| 115 | CGCCTCAA |
| 116 | CGCGGAAT |
| 117 | CGCGTTAC |
| 118 | CGGAAGGA |
| 119 | CGGACTCT |
| 120 | CGGTGAGA |
| 121 | CGGTTGTT |
| 122 | CGTAACAC |
| 123 | CGTAGCTT |
| 124 | CGTCTATG |
| 125 | CGTTCTCG |
| 126 | CTACCGGA |

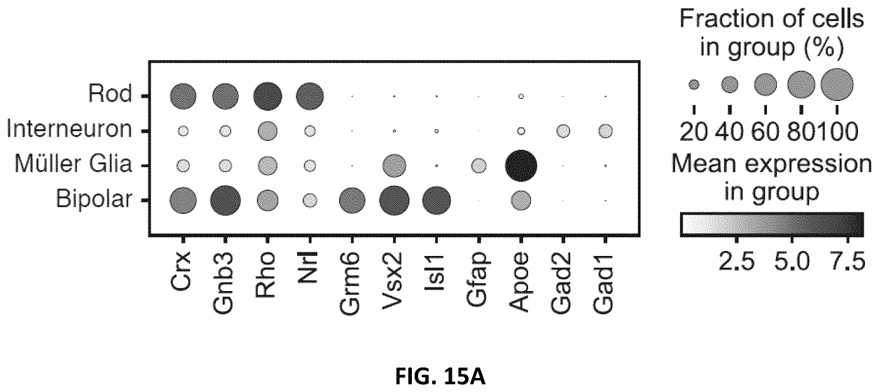

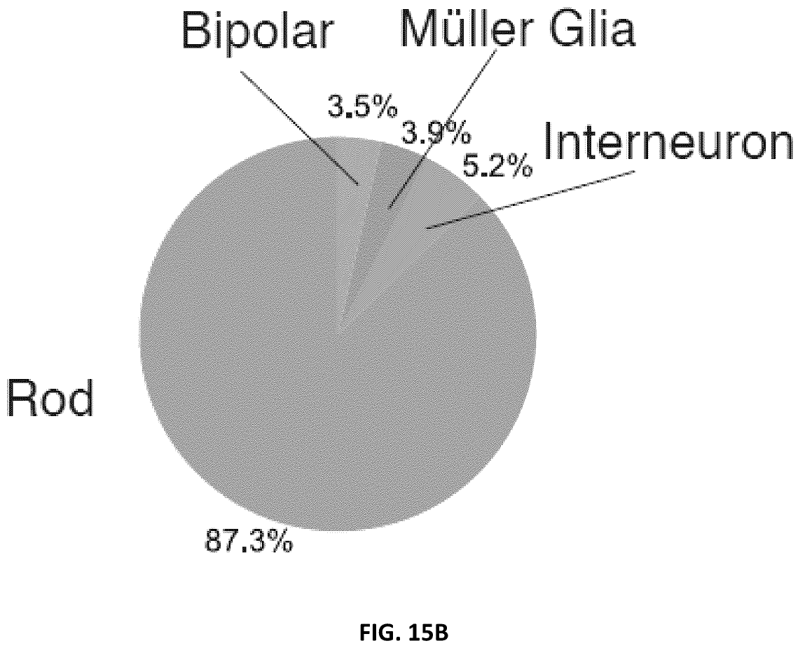

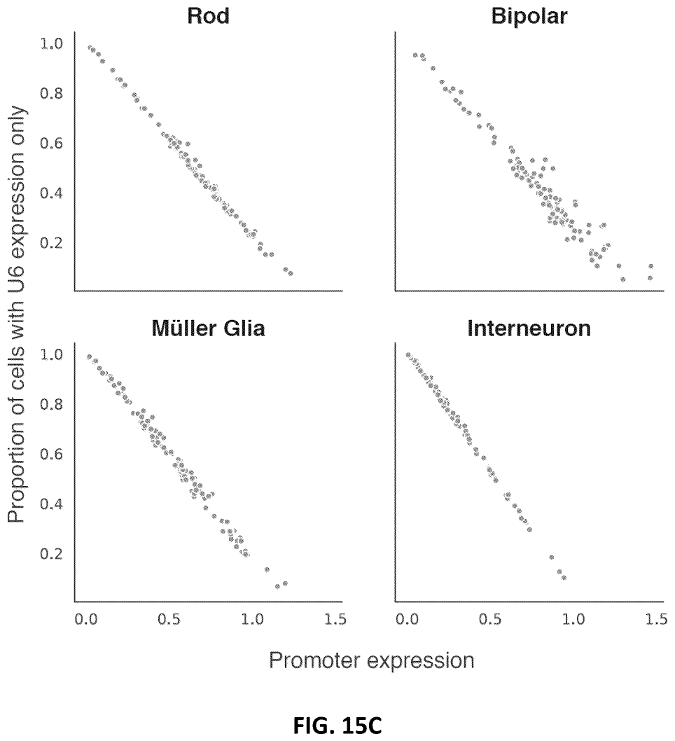

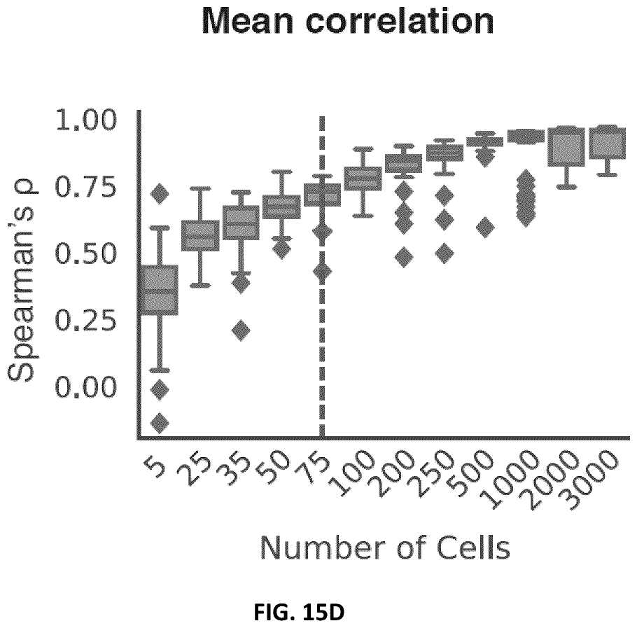

The Gnb3 promoter variant library was introduced into newborn mouse retinas and assessed the cell types into which the library entered by scRNA-seq (FIG. 10C). A total of 22,161 cells were obtained from two replicate experiments with a mean of 22,528 reads per cell and 1,642 genes per cell. The scRNA-seq data showed that we recovered rod photoreceptors (87.3%), bipolar cells (3.5%), interneurons (i.e. amacrine cells) (5.2%), and Willer glia cells (3.9%) (FIGS. 10D, 15A, and 15B).

The expression of each Gnb3 promoter variant in each cell type was then computed by sequencing the Gnb3-expressed barcodes and the U6 barcodes from single cells. Cells with U6-expressed cBC counts, but no Gnb3-expressed cBC counts, represented cells in which that promoter variant was silent. On average, Gnb3 promoter variants were silent in 22% of cells, but this number varied widely (FIG. 10E) and was linearly related to the strength of the promoter, with stronger promoters expressing in a larger fraction of cells (FIG. 15C). Using both the Gnb3-driven and U6-driven counts allowed one to compute the average expression of a promoter variant across all the cells of a given cell type, while still accounting for cells in which that promoter variant is silent (Methods).

Biological replicates measurements of the Gnb3 promoter variant library were reproducible in all four cell types (FIG. 15C). Reproducibility was highest in rod cells (Spearman's p: 0.97, Pearson's R: 0.98) because rod cells are the most abundant cell type in the mouse retina. The reproducibility was slightly lower in the rarer cell types (bipolar cells: Spearman's p: 0.88, Pearson's R: 0.92, Willer glia: Spearman's p: 0.93, Pearson's R: 0.95, and interneurons: Spearman's p: 0.95, Pearson's R: 0.98), but remained high enough to assess the expression of individual library members. How reproducibility scales with the number of cells in scMPRA was determined by subsampling the expression data. The minimum number of cells required for reproducible measurements (Spearman's p>0.75) of mean reporter gene levels is 75 cells (FIG. 15D). The results show that scMPRA works well for measuring reporter gene levels across cell types in complex tissues using small numbers of cells

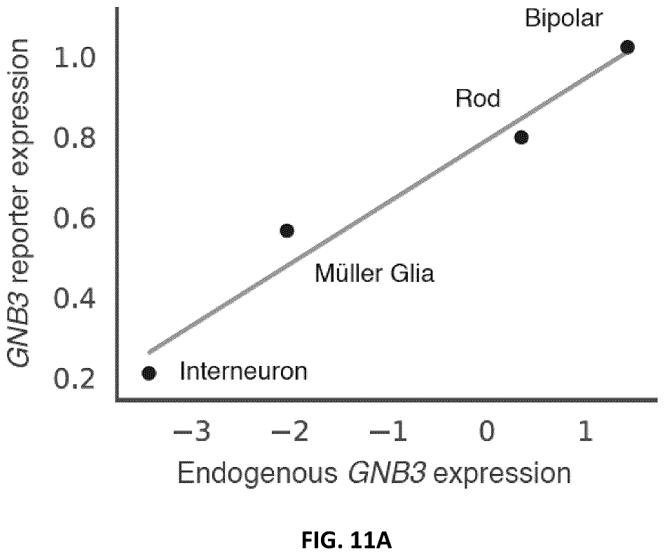

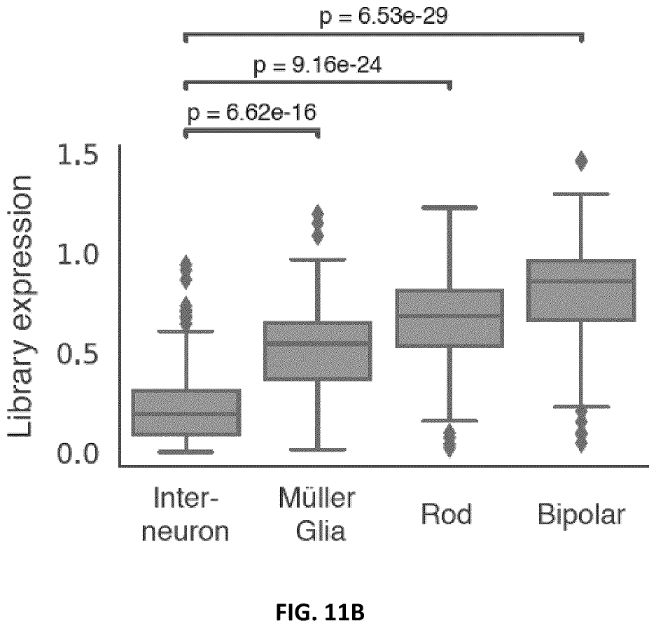

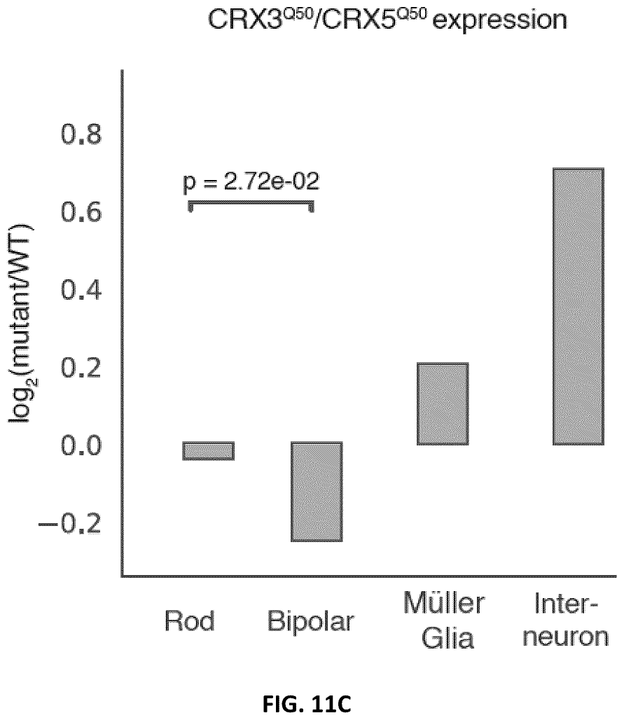

Two additional observations suggest that scMPRA measurements are accurate in ex vivo retinas. First, the expression of the wild type Gnb3 reporter, as well as the average expression of all Gnb3 promoter variants, correlates with endogenous Gnb3 expression in the corresponding scRNA-seq data (FIGS. 11A and 11B). Second, the scMPRA data reproduced the known effect of a cell type specific Gnb3 promoter variant. Murphy et al. showed that altering two of the K50 homeobox sites in the Gnb3 promoter to Q50 sites reduces expression in bipolar cells while leaving expression in rod cells relatively unaffected. The same reduction in bipolar cells was observed when compared with rod cells for this same mutant (FIG. 11C). In addition, scMPRA also revealed that this mutant shows increased activity in Willer glia and interneurons. Taken together, these observations demonstrate that scMPRA is reproducible and accurate when applied to cell types in a complex tissue.

scMPRA Reveals Cell-Type Specific Promoter Variants.

The Gnb3 library was designed to probe components of the promoter including five binding sites for K50-type homeodomain TFs, an E-box binding site, and two evolutionarily conserved regions (FIG. 12A). In this experiment the effect of a mutation is defined as its relative 4 fold-change to the WT Gnb3 promoter in each cell type because the Gnb3 promoter is expressed at different levels across cell types (FIG. 11A). The homeobox sites were labeled as Cone Rod Homeobox (CRX) sites because CRX is a K50-homeodomain protein that plays an important role in rods and bipolar cells and is required for Gnb3 expression. K50-homeodomains contain lysine at the 50th amino acid residue and have different binding preferences from Q50-homeodomains, which contain glutamine at position 50 and are also expressed in the retina.

Inactivating mutations in any individual CRX site decreased Gnb3 reporter expression in bipolar and rod cells, but deletion of either CRX1 or CRX5 also resulted in increased expression in interneurons (FIG. 12B). The CRX2 disruption had the largest effect on expression, and mutating the CRX2 site in combination with any other CRX site also caused large reductions in expression in rods and bipolar cells. Murphy et al. previously reported that different retinal cell types differ in their usage of K50 vs Q50 motifs, suggesting that promoters containing K50 or Q50 motifs may display cell-type specific differences. Single and double swaps of K50 CRX binding sites with Q50 binding sites tended to yield cell-type specific effects, primarily because interneurons displayed larger responses to the Q50 swaps compared with rod and bipolar cells (FIG. 12C). Increasing the affinity of CRX sites tended to have mild effects on expression in rods and bipolar cells, but increased expression significantly in interneurons (FIG. 12D). The results from modifying CRX sites demonstrated that perturbations to single binding sites can produce cell-type specific effects.

Next, the effects of single nucleotide changes in the E-box binding site were examined (FIG. 12E). Helix-Loop-Helix (bHLH) transcription factors, which bind E-box motifs, are critical for the development of multiple retinal cell types. Several single-nucleotide substitutions in the E-box resulted in strong effects on expression, although only one substitution produced significant cell-type specific effects. While the E-box is critical for strong expression of the Gnb3 promoter, subtle changes to its sequence do not generally result in cell-type specific changes to its activity.

To examine the effects of more severe sequence changes, and to assess the effects of perturbations outside the known TFBSs, mutations were tiled through the two evolutionarily conserved regions shuffling 5 bp at a time (FIG. 12F). Mutations in all six TFBSs resulted in cell-type specific changes in expression, but several mutations in the Gnb3 promoter outside of the known TFBS also resulted in cell-type specific changes in expression. Thus, other information in the Gnb3 promoter provides important cell-type context for the functioning of the CRX and E-box motifs.

The analysis of the Gnb3 promoter shows that single-binding site and single-nucleotide variants can result in cell-type specific changes to cis-regulation and that scMPRA is a powerful tool for identifying these changes across cell-types in mammalian tissues. The cis-regulatory logic of the Gnb3 promoter keeps it expressed at high levels in rods and bipolar cells in the early postnatal period, and at much lower levels in interneurons, which may be why most cell-type specific perturbations result in effects of different sizes in interneurons when compared with rods and bipolar cells.

### DISCUSSION

Herein, a single-cell MPRA method to measure the cell-type and cell-state specific effects of CRSs is presented. It has been demonstrated that scMPRA detects cell-type specific reporter gene activity in a mixed population of cells as well as in living retinal tissue, and cell-state specific activity in isogenic K562 cells. The assay is reproducible and reports accurate mean levels of reporter gene activity in as few as 75 cells in a complex tissue. New methods that increase the number of single cells measured per experiment will increase the size of libraries that can be assayed by scMPRA. The dynamic range was relatively small in this study (8-fold between the strongest and weakest Gnb3 variants), which may reflect the activity of these specific sequences, but may also arise from the low efficiency of mRNA capture in single cells. scMPRA will therefore benefit from continuing improvements of methods to capture and recover mRNA from single cells.

The success of an scMPRA experiment will depend at least in part on the efficiency of delivering DNA to the relevant cell types. For tissues with low transfection or transduction efficiencies, most cells will not contain a library member and will therefore be uninformative. This is a problem because of the limited number of cells that can be sequenced with current scRNA-seq protocols. Likewise, if the relevant cell type is rare in the tissue of interest then some enrichment may be necessary to obtain enough cells to make robust measurements. Thus, scMPRA will work best in systems amenable to high efficiency transfection or transduction. This consideration motivated our choice of the retina as a test system for scMPRA because DNA can be delivered to a large fraction of the cells in an ex vivo retina with high efficiency.

With the burgeoning of Adeno-associated viral delivery systems, it is anticipated that the efficiency of DNA delivery will gradually improve for many tissues and systems. Coupling AAV-based methods with scMPRA will allow it to be widely used to study cis-regulatory effects in a variety of complex tissues. Given the hypothesis that non-coding variants with cell-type specific effects underlie a large fraction of human disease, an important application of scMPRA will be to test polymorphisms identified in human genetic studies for cell-type specific cis-regulatory effects.

### Methods

### Cell Culture

K562 cells were obtained from the Genome Engineering & iPSC Center at Washington University School of Medicine. HEK293 cells (ATCC CRL-1573) were purchased from ATCC (American Type Culture Collection). Cell lines were tested for Mycoplasma and were negative. K562 cells were cultured in Iscove's Modified Dulbecco's Medium (IMDM, Gibco 12440046)+10% Fetal Bovine Serum (FBS, Gibco 10438034)+1% non-essential amino acids (NEAA, Gibco 11140050)+1% Penicillin-Streptomycin (Gibco 15140122) at 37° C. with 5% CO₂. HEK293 cells were cultured in Eagle's Minimum Essential Medium (EMEM, ATCC #30-2003)+10% Fetal Bovine Serum (FBS, Gibco 10438034))+1 pen/strep (Gibco 15140122) at 37° C. with 5% CO₂.

### Core Promoter Library Cloning

A two-level barcoding strategy to enable single-cell normalization of plasm id copy number has been developed. This strategy was applied to a library of core promoters previously tested by bulk MPRA. That core promoter library contains 676 core promoters, each with a length of 133 bp. The library cloning was done in three steps: First, a library of 676 core promoters each barcoded with 10 different cBCs was synthesized and this library was cloned into a backbone. In a second step, a dsRed fluorescent reporter cassette was cloned between each core promoter and its associated cBCs as described. Thirdly, this library for scMPRA was modified by adding random barcodes downstream of the cBCs, but upstream of the polyA site.

To add the random barcodes (rBCs) a single-stranded 90 bp DNA oligonucleotide (oligo) containing a 25 bp random sequence (the rBC), a restriction site, and 30 bp homology to the library vector on each side of the rBC region was synthesized (SEQ ID NO:11). NEBuilder® HiFi DNA Assembly Master Mix (E2621) was used to clone this oligo into the core promoter library. 4 μg of the plasm id library was split into four reactions and digested with 2 μL of Sall for 1.5 hours at 37° C. The digested product was purified with the Monarch Gel Extraction Kit (NEB T1020). The insert single-stranded DNA was diluted to 1 μM with H₂O. Three assembly reactions were pooled together, each reaction containing 100 ng of digested library backbone, 1 μM of insert DNA, 1 μL of NEBuffer 2, 10 μL of 2X HiFi assembly mix, and H₂O up to 20 μL. The reaction was incubated at 50° C. for 1 hour. The assembled product was purified with the Monarch PCR&DNA Cleanup kit (NEB T1030) and eluted in 12 μL of H₂O.

The assembled plasmid was transformed using Gene Pulser Xcell Electroporation Systems by electroporation (Bio-Rad 1652661) into 50 μL of ElectroMax DH10B electrocompetent cells (Invitrogen 18290015) with 1 μL of assembled product at 2 kV, 2000 Ω, 25 nF, with 1 mm gap. 950 μL of SOC medium (Invitrogen 15544034) was added to the cuvette and then transferred to a 15 mL Falcon tube. Two transformations were performed, and each tube was incubated at 37° C. for 1 hour on a rotator with 300 rpm. The culture was then added to pre-warmed 150 μL LB/Amp medium and grown overnight at 37° C. 1 μL of the culture was also diluted 1:100 and 50 μl of the diluted cultured was plated on an LB agar plate to estimate the transformation efficiency. For the core promoter library, DNA from more than 4×10⁸ colonies was prepared. Shallow sequencing of this library (below) showed that the majority of library members encoded unique cBC-rBC combinations.

### Gnb3 Promoter Variant Library Design and Cloning

The Gnb3 library was designed to probe components of the promoter including five binding sites for K50-type homeodomain TFs, an E-box binding site, and two evolutionarily conserved regions. The K50 homeobox sites as Cone Rod Homeobox (CRX) sites were labeled because CRX is a K50-homeodomain protein required for Gnb3 expression and a key-lineage determining factor in retina, even though other K50-type homeobox proteins are also expressed in retinas. To test whether the disruption of CRX sites in the Gnb3 promoter has cell-type specific effect, the following three types of mutations were made: (1) All individual and pairwise deletions of the CRX binding sites by mutating the CRX sites to 5′-CTACTCCC-3′. (2) All individual and pairwise mutations of CRX binding sites from K50 homeobox to Q50 homeobox motifs: 5′-CTAATTAC-3′. (3) All individual mutations of CRX binding sites to high (5′-CTAATCCC-3′), medium (5′-CTAAGCCC-3′) and low affinity (5′-CTTATCCC-3′) K50 homeobox sites. Our unpublished data suggested that the E-box is important for the Gnb3 promoter activity and E-box motif is bound by many neuronal specific TFs, hence each base pair in the E-box was mutated to every other base pair and pairwise mutations of the two core base pairs in the E-box motif were made. Lastly, an unbiased approach was taken to screen for potential cell-type-specific mutations by shuffling mutations across the two conserved regions in the Gnb3 promoter. Each conserved region was tiled into 5 bp windows and the nucleotides within each window were shuffled.

The library of Gnb3 promoter variants was constructed in four steps. In the first step, the Gnb3 promoter variant library was cloned into the core promoter library vector backbone. Double-stranded DNA fragments were ordered from Integrated DNA Technologies encoding the varying part of the (520 bp) Gnb3 promoter and 113 promoter variants. The wild-type Gnb3 promoter sequence was included twice, each time fused to a different cBC. The DNA fragments were manually pooled and cloned together as a library. In the second step, the remaining Gnb3 promoter (300 bp) and a mEmerald reporter cassette were cloned between the Gnb3 promoter variants and the first cBC copy using HiFi assembly. In the third step, NEB HiFi DNA Assembly Master Mix (NEB E2621) was used to insert the U6 promoter between the two copies of the cBCs where it drives expression of the downstream copy of the cBC. In the fourth step, high-complexity rBCs were introduced between the first cBC and the U6 promoter. A DNA oligo was synthesized containing a 25 bp random sequence (the rBC), a restriction site, and 30 bp homology to the library vector on each side of the rBC barcode region. HiFi Assembly was then used to clone the rBC oligos into the Gnb3 promoter variant library. In this final library, each plasmid contains a Gnb3 promoter variant driving mEmerald with a unique cBC-rBC combination in its 3′ UTR, which is followed by a polyA signal and the U6 promoter driving a second copy of the cBC, a capture sequence, and a termination signal. A total of eight HiFi Assembly reactions were pooled together to increase the library complexity. This library was transformed and amplified in E. coli as described above, and DNA was prepared from 2×10⁹ colonies.

### Estimating Library Complexity

To estimate the complexity of the core promoter library, the DNA library was sequenced using a nested PCR-based Illumina library preparation protocol. Briefly, Q5 polymerase (NEB M0515) was first used to amplify the region containing the two barcodes with SCARED 17 and SCARED P18 (SEQ ID NOS:1-2). The total reaction volume was 50 μL using 50 ng of plasmid library with 2.5 μL of 10 μM primer each. After 25 cycles of amplification (61° C. annealing temperature, 30 s extension time) the product was purified with the Monarch PCR&DNA Cleanup kit (NEB T1030) and eluted with 20 μL of ddH₂O. For the second round of PCR, the primers SCARED P19 and SCARED P20 (SEQ ID NOS:3-4) were used in a 25 μL reaction with 0.25 μL product from the previous step (61° C. annealing temperature, 30 s extension time). After 10 cycles of amplification, the product was purified using the Monarch PCR&DNA Cleanup kit (NEB T1030). For the last PCR, the P5 and P7 Illumina adapters were added with SCARED P5, and SCARED P7 (SEQ ID NOS:5-6) with 10 cycles of amplification in a 25 μL reaction with 2 μL of purified product (65° C. annealing temperature, 30 s extension time). This final product was sequenced on an Illumina MiSeq, and a total of 1,693,933 reads were obtained. After filtering out reads without a cBC or rBC of the correct length, a total of 1,359,176 reads (80% of the total reads) were obtained, and 99.5% represented unique cBC-rBC pairs. For the Gnb3 library, shallow sequencing was performed, and a total of 1,939,479 reads were obtained. After filtering out reads without correct cBC or rBC, a total of 1,838,415 reads (94.7% of the total reads) were obtained. Among the 1,838,415 correct reads, 99.5% represented unique cBC-rBC pairs.

Estimating the Probability of Identical cBC-rBC Pairs in the Same Cell

The probability that more than one copy of a plasm id carrying the same cBC-rBC pair would be transfected into the same cell was estimated. This probability is defined as the collision rate. If the library is transfected into n cells, and a specific cBC-rBC pair is present at m copies in the library, then the expected number of collisions per experiment is given by:

$${n}^{- m} {\sum }_{k = 0}^{n} ( \begin{matrix}n \\ k\end{matrix} ) {\sum }_{q = 0}^{( n - k )} ( \begin{matrix}n - k \\ q\end{matrix} ) ( \begin{matrix}m \\ q\end{matrix} ) q ! {\{ \begin{matrix}m - q \\ n - k - q\end{matrix} \}}_{n \geq 2} ( n - k - q ) ! ( m - q )$$

where k denotes the number of cells that received no plasmid, q denotes the number of cells transfected with exactly one plasmid, parentheses denote the binomial coefficient, and brackets denote the partition function. The above expression was simplified by substituting with the bivariate generating function, and the expected number of collisions per experiment is:

$$m ( 1 - {( \frac{n - 1}{n} )}^{m - 1} )$$

The expected number of collisions per cell (λ) is given by,

$$λ = \frac{m ( 1 - {( \frac{n - 1}{n} )}^{m - 1} )}{n}$$

And, assuming collisions are a Poisson process, the probability of at least one collision in a cell is:

P(Collision)=1−e^{-λ}

Using this framework, the probability of a collision in the experiment can be estimated. We assume one million cells (n) are transfected using 10 μg of plasm id DNA, and that the effective number of plasm ids that enter the nucleus is 10% of that input amount (1 μg) 1 μg of plasmid DNA is 2.3×10¹¹. Thus, the value of m in the nucleus is 2.3×10 11 divided by the number of unique members of the library. This allows one to calculate P(Collision) for a library of any given size. This framework shows that a library with 1.6×10⁸ unique members is required to achieve P(Collision)=0.01. To be 99% sure that a library has at least 1.6×10⁸ unique members requires preparing that library 4.5 times as many independent colonies (7.2×10⁶), assuming a Poisson distributed library. The core promoter library was prepared from 4×10⁸ colonies, 55 times more than required for P(Collision)=0.01, and the Gnb3 variant library was prepared from 2×10⁹ colonies, 277 times more than required for P(Collision)=0.01.

### Cell Line Transfections

K562 cells were transfected with the core promoter library using electroporation with the Neon transfection system (Invitrogen MPK5000). One million cells were transfected with 2 μg of plasmid DNA (mixed-cell experiment) or 10 μg of plasmid DNA (K562 sub-state experiment), with 3 pulses of 1450 V for 10 ms.

HEK293 cells were transfected with the core promoter library using the Lipofectamine3000 reagent (Invitrogen L3000001) following the manufacturer's protocol. 4 μL of p3000 reagent, 4 μL of Lipofectamine, and OptiMEM were mixed with 2 μg of plasmid DNA to a volume of 250 μL. The lipofectamine reagents and plasmid were mixed and incubated at room temp for 15 minutes and then added dropwise to the cells. K562 and HEK293 cells were harvested 24 hours after transfections for scMPRA.

### Ex Vivo Culturing and Transfection of Mouse Retinas

CD-1 IGS mice were obtained from Charles River Laboratory. Retinas from newborn (P0) mice were dissected and electroporated. The sex of the mice could not be determined at the P0 stage. Retinas were dissected in serum free medium (SFM; 1:1 DMEM:Ham's F12 (Gibco 11330-032), 100 units/mL penicillin and 100 μg/mL streptomycin (Gibco 15140-122), 2 mM GlutaMax (Gibco 35050-061) and 2 μg/mL insulin (Sigma 16634) from surrounding sclera and soft tissue leaving the lens in place. Retinas were then transferred to an electroporation chamber (model BTX453 Microslide chamber, BTX Harvard Apparatus modified) containing 0.5 μg/μL of the Gnb3 promoter variant library 0.5 μg/μL of a plasm id in which the Rhodopsin promoter drives the dsRed fluorophore. For each replicate experiment, three retinas were electroporated. Five square pulses (30 V) of 50-ms duration with 950-ms intervals were applied using a pulse generator (model ECM 830, BTX Harvard Apparatus.). Electroporated retinas were removed from the electroporation chamber and allowed to recover in SFM for several minutes before being transferred to the same medium supplemented with 5% fetal calf serum (Gibco26140-079). The retinas were then placed (lens side down) on polycarbonate filters (Whatman, 0.2 μm pore size 110606) and cultured at 37° C. in SFM supplemented with 5% fetal calf serum for 8 days.

Electroporated retinas were harvested and dissociated with modifications as outlined below. Briefly, three retinas/replicate were washed 3× in cold Hanks' Balanced Salt Solution (HBSS) (Gibco 14025-076) and were then incubated in 400 μL of HBSS containing 0.65 mg papain (Worthington Biochem LS003126) for 10 min at 37° C. 600 uL of Dulbecco's Modified Eagle Medium (DMEM) (Gibco 11965-084) containing 10% fetal calf serum (FCS) (Gibco 26140-079) was added and the tissue was gently triturated with P1000 to achieve single cells suspension. 100 units of DNase1 (Roche 04716728001) were added to the cell suspension and incubated an additional 5 min at 37° C. Cells were centrifuged at 400 g for 4 min then resuspended in 600 mL of sorting buffer (2.5 mM EDTA (Sigma EDS), 25 mM HEPES (Sigma H3375), 1% BSA (Sigma H3375) in HBSS) and passed through a 35 μm filter and used directly for Fluorescence Activated Cell Sorting (FACS).

Because the majority of cells in murine retinas are rod photoreceptors, other cell types were attempted to be enriched using FACS. The co-electroporated Rhodopsin-DsRed construct marks rod cells specifically. Therefore, FACS was used to generate a 1:1 mixture of dsRed+ to dsRed− cells from dissociated retinas. This procedure should yield a mix of cells in which rod cells comprise 50% of the total cells. In practice, rod cells still comprised 87% of the cells that were analyzed by scMPRA.

Bulk MPRA from Cell Lines

For both K562 cells and HEK293 cells, the promoter library was transfected as described above, total mRNA was extracted, and reverse transcription was performed using the Superscript IV Reverse Transcriptase Kit (Invitrogen 18090010). Sequencing libraries were then constructed using the same method of library preparation described above in Estimating Library Complexity from the cDNA and the plasm id library used for transfection. The resulting libraries were sequenced on an IIlumina MiSeq instrument. The barcodes were extracted from the reads and tabulated for the RNA and DNA pools respectively. The activity of each library member was computed as log 2(RNA counts/DNA counts). The activities of barcodes linked to the same core promoter were averaged to calculate the final activity of each promoter.

Single-Cell RNA-Seq for scMPRA

To perform scMPRA 2000 cells from the HEK/K562 mixed pool per replicate for each mixed cell experiment, 2500 cells per replicate for the K562-only experiment and 2500 cells (after sorting) per replicate for the retina experiment were targeted. The cells were prepared according to the manufacturer's instructions for the 10× Chromium Single Cell 3′ Feature Barcode Library Kit (PN-1000079), with the changes made that are detailed below.

The goal was two-fold: to quantify the cBC-rBC pairs from each single cell and to sequence the cellular mRNAs from those same single cells. All polyadenylated RNAs (barcoded reporter RNAs and cellular mRNAs) were captured from single cells following the manufacturer's protocol up to the cDNA amplification step.

For the cellular mRNAs (transcriptome), the 10× protocol was followed, using ¼ of the cDNA library to generate dual-indexed transcriptomes. To quantify the cBC-rBC pairs, separate PCRs were performed using primers specifically targeting the reporter gene to improve barcode recovery efficiencies. Because the 10× protocol only uses ¼ of the generated cDNA, the barcodes from another ¼ of the pellet cleanup were separately amplified. Q5 polymerase (NEB M0515) was used to amplify the region containing the cBC-rBC pairs with SCARED P17 and SCARED P18 with 10 cycles (61° C. annealing temperature, s extension time). The sample was divided equally into eight PCR reactions, each with 50 μL of total volume to reduce possible jackpotting. The product was then purified with the Monarch PCR&DNA Cleanup kit (NEB T1030) and eluted with 20 μL of ddH₂O. Sequencing adapters were then added using an additional two rounds of PCR. The first adapter PCR was performed with SCARED P21 and SCARED PP2 with a total of 10 ng of product from the barcode PCR (61° C. annealing temperature, 30 s extension time). Again, eight PCR reactions were pooled, each with 50 μL of total volume and 10 PCR cycles. The PCR product was purified using the Monarch PCR&DNA Cleanup kit (NEB T1030). For the last PCR, to add the P5 and P7 IIlumina adapters, the primers SCARED P45 and SCARED PP3 (SEQ ID NOS:9-10) with 10 ng of product were used and eight PCR reactions were pooled, each with 50 μL of total volume and 10 PCR cycles (58° C. annealing temperature, 30 s extension time).

For the U6 promoter library construction, Step 4 of the 10× feature barcoding library preparation protocol (Chromium Next GEM Single Cell 3′ Reagent Kits v3.1 (Dual Index) CG000316 Rev C) was followed as written.

The transcriptome and barcode libraries were mixed in equimolar ratios and paired end sequencing was performed on the IIlumina NextSeq 500 with 28×105 paired-end reads. Read1 was limited to 28 bp to avoid sequencing the constant poly(A) sequence.

scRNA-Seq Data Processing

The single-cell RNAseq data were processed using Cellranger 6.0.1 (https://github.com/10xGenomics/cellranger) and Scanpy 1.8.1 (https://github.com/theislab/scanpy) following the standard pipeline. Briefly, different sequencing runs from the same biological replicate were pooled together and processed with CellRanger 6.1.1; the final output expression matrix was then imported into Scanpy for further processing. Cells with less than 1000 genes, genes that were present in less than three cells, and cells with high counts of mitochondrial genes were first removed. Next, the UMI counts were normalized to the total cell UMI counts. The normalized expression matrix was used for clustering and visualization with Scanpy.

scMPRA Data Processing

For each promoter library, paired-end reads generated from barcoded reporter RNAs were processed with custom scripts that can be found on GitHub (https://github.com/barakcohenlab/scMPRA). In each paired-end read, Read1 contains a 10× cell barcode and a UMI, while Read2 contains the cBC and rBC sequences. A “quad” is defined as a 10× Cell Barcode, UMI, cBC, and rBC originating from the same individual paired-end read. To tabulate the cBC-rBCs the constant sequences flanking both barcodes were first matched and reads where either barcode was not the correct length were filtered out. This filtering was performed using a stand-alone program (https://github.com/szhao045/scMPRA_parsingtools). Second, incorrect 10× Cell Barcodes were filtered out based on the CellRanger output barcode list using error-correction with a maximum Hamming distance of one. Third, to mitigate the effect of template-switching during the PCR steps, the rank read depth for each unique quad was plotted and an “elbow point” was identified at a minimum depth of 1 read for the mixed-cell and the retina experiment, and 10 reads for the K562 alone experiment. All reads above the minimum depth were kept and a low-depth unique quad was kept if it contained a cBC-rBC matching a high-depth pair with a Hamming distance of at most one. Lastly, for the mixed-cell experiment and the K562 cell alone experiment, any cell with less than 100 scMPRA-associated UMIs was removed, since the scMPRA reads from those cells were poorly sampled. For the last step, because the retina experiment contains additional information from the U6 promoter, thresholding was not performed based on cells. Since U6 promoter data provides information on whether a given cBC in a given cell is sampled well, all unique barcode pairs containing only 1 UMI for a cBC were removed.

### Calculating the Single-Cell Activities of Promoters

Once the high-confidence quads were identified, A, the activity of a promoter in an individual cell, was computed using,

$$A = \frac{{\sum }_{i = 1}^{n} UMI count for {cBC}_{i}}{{\sum }_{i = 1}^{n} rBC count for {cBC}_{i}}$$

where n is the number of unique cBCs that mark a single promoter in the library, and the UMI and rBC counts are summed over all quads with a given 10× cell barcode. C, the cell-type specific activity of a promoter, is then computed as,

$$C = \frac{{\sum }_{j = 1}^{m} {A}_{j}}{m}$$

where m is the number of cells in a given cell type, and all 10× cell barcodes assigned to a given cell type are identified from their matched scRNA-seq profiles. For scMPRA data from the retina, the equation for cell-type specific activity was modified as follows,

$$C = \frac{{\sum }_{j = 1}^{P} {A}_{j}}{P + U}$$

where P is the number of cells of a given cell type in which Gnb3-driven cBCs were detected and U is the number of cells of that cell type for which a U6 promoter cBC was detected without detecting any corresponding Gnb3-driven cBC. This modification has the effect of adding activities of zero for all cells with U6-driven cBCs that did not express a Gnb3-driven cBC.

### Cell Cycle Analysis

Cell cycle analysis for the scRNA-seq experiment was done with Scanpy 1.8.1 with cell cycle genes. The expression profile of each cell was projected onto a PCA plot based on the list of cell cycle genes using Scanpy.

### Motif Analysis

The core promoters were first clustered according to their expression levels in the different cell sub-state populations by hierarchical clustering. We categorized the data into up/down regulated clusters at the first branching point, aiming to preserve the large structure. Core promoter motifs in each promoter were then identified using the parameters for each motifs position weight matrix (PWM) with MAST v4.10.048 and the proportion of promoters containing each motif in each promoter class was plotted.

### Statistical Analyses

All statistical analyses were done using Python 3.9.6, Numpy 1.12.149, Scipy 1.6.3, and R 4.0.2. For all boxplots presented herein, the bounds of the box represent the upper and lower quartiles respectively, and the center line represents the median. The whiskers extend to the maxima/minima except for points determined to be outliers using a method that is a function of the interquartile range.

1. A plurality of expression vectors, wherein each expression vector of the plurality comprises a first identifying nucleic acid barcode (rBC) uniquely associated with the individual expression vector.

2. The plurality of expression vectors of claim 1, wherein the first identifying nucleic acid barcode (rBC) is a randomized sequence.

3. The plurality of expression vectors of claim 2, wherein each expression vector further comprises:
a. a nucleic acid regulatory element;
b. an open reading frame optionally encoding a reporter gene; and
c. a second identifying nucleic acid barcode (cBC) uniquely associated with the nucleic acid regulatory element;
wherein the nucleic acid regulatory element of each expression vector is selected from a plurality of different nucleic acid regulatory elements.

4. The plurality of expression vectors of claim 3, wherein each nucleic acid regulatory element is a genetic variant of a single nucleic acid regulatory element.

5. The plurality of expression vectors of claim 4, wherein each nucleic acid regulatory element differs from the remaining nucleic acid regulatory elements by at least one nucleotide substitution, deletion, or insertion.

6. The plurality of expression vectors of claim 5, wherein the regulatory element is a cis-regulatory element.

7. The plurality of expression vectors of claim 6, wherein the cis-regulatory element is an enhancer, promoter, insulator, or silencer.

8. The plurality of expression vectors of claim 7, wherein the cis-regulatory element is a core promoter.

9. The plurality of expression vectors of claim 1, wherein each expression vector further comprises a cell barcode or a UMI sequence.

10. The plurality of expression vectors of claim 9, wherein the cell barcode comprises a 10× cell barcode and the UMI sequence comprises a 10× UMI sequence.

11. The plurality of expression vectors of claim 1, wherein each expression vector further comprises a capture sequence or a polyadenylation signal.

12. The plurality of expression vectors of claim 3, wherein the nucleic acid regulatory element and the cBC are linked.

13. The plurality of expression vectors of claim 12, wherein the nucleic acid regulatory element and cBC are linked through a process selected from synthesis, ligation, PCR, and any combination thereof.

14. A method for determining individual activities of a plurality of nucleic acid regulatory elements, the method comprising:
a. introducing a plurality of expression vectors into a population of cells, wherein each expression vector of the plurality comprises a first identifying nucleic acid barcode (rBC) uniquely associated with the individual expression vector;
b. performing single-cell RNA sequencing (scRNAseq) on the population of cells; and
c. quantifying expression of cBC and/or rBC in an individual cell;
wherein the amount of each cBC detected indicates the activity of the associated regulatory element in the cell and the amount of each rBC detected indicates the number of expression vectors comprising the associated regulatory element in the cell.

15. The method of claim 14, further comprising generating a scRNAseq profile for the individual cell, wherein the scRNAseq profile identifies the cell type of the individual cell.

16. The method of claim 14, wherein the population of cells comprises cells in different biological states, the different biological states comprise different stages of cell cycle, different subpopulations of same cell type, or a combination thereof.

17. The method of claim 14, wherein the population of cells comprises multiple cell types.

18. The method of claim 14, further comprising normalizing the activity of the regulatory element to the number of expression vectors comprising the regulatory element in the cell.

19. A method for identifying a regulatory element having cell type-specific activity, the method comprising:
a. introducing the plurality of expression vectors into a population of cells, wherein each expression vector of the plurality comprises a first identifying nucleic acid barcode (rBC) uniquely associated with the individual expression vector;
b. performing single-cell RNA sequencing (scRNAseq) on the population of cells;
c. quantifying expression of cBC and/or rBC in an individual cell, wherein the amount of each cBC detected indicates the activity of the associated regulatory element in the cell; and the amount of each rBC detected indicates the number of expression vectors comprising the associated regulatory element in the cell;
d. generating a scRNAseq profile for the individual cell, wherein the scRNAseq profile identifies the cell type of the individual cell; and
e. determining the regulatory element to have cell type-specific activity if the activity of the regulatory element differs substantially between at least two cell types.

20. A method for determining variance in activity of a plurality of nucleic acid regulatory elements, the method comprising:
a. introducing the plurality of expression vectors into a population of cells, wherein each expression vector of the plurality comprises a first identifying nucleic acid barcode (rBC) uniquely associated with the individual expression vector;
b. performing single-cell RNA sequencing (scRNAseq) on the population of cells;
c. quantifying expression of cBC and/or rBC in an individual cell; wherein the amount of each cBC detected indicates the activity of the associated regulatory element in the cell, and the amount of each rBC detected indicates the number of expression vectors comprising the associated regulatory element in the cell; and
calculating the variance in activity of the regulatory element across the population of cells.
